(19) United States
(12) Patent Application Publication
(10) Pub. No.: US 2022/0176096 A1
(43) Pub. Date: Jun. 9, 2022

(54) MICRONEEDLES TO DELIVER THERAPEUTIC AGENT ACROSS MEMBRANES

(71) Applicant: THE TRUSTEES OF COLUMBIA UNIVERSITY IN THE CITY OF NEW YORK, NEW YORK, NY (US)

(72) Inventors: Aykut AKSIT, New York, NY (US); Anil K. LALWANI, New York, NY (US); Jeffrey W. KYSAR, New York, NY (US); Alan WEST, New York, NY (US)

(21) Appl. No.: 17/675,440

(22) Filed: Feb. 18, 2022

Related U.S. Application Data
(63) Continuation-in-part of application No. PCT/US20/49638, filed on Sep. 8, 2020.
(60) Provisional application No. 62/898,312, filed on Sep. 10, 2019.
(60) Provisional application No. 63/150,632, filed on Feb. 18, 2021.

(51) Int. Cl. A61M 37/00; C25D 1/02

(52) U.S. Cl. CPC A61M 37/0015; C25D 1/02; A61M 2037/0023; A61M 2037/003; A61M 2210/0662; A61M 2037/0046; A61M 2205/583; A61M 2037/0007; A61M 2037/0053

(57) ABSTRACT

The disclosed subject matter relates to a system and method for delivery of therapeutic agents across membranes such as to the inner ear. The system includes a plurality of microneedles that can be delivered to the round window membrane by a delivery device, e.g. catheter, and is capable of controlled penetration of the round window membrane to create temporary and self-closing perforations.

### CROSS-REFERENCE TO RELATED APPLICATIONS

This application claims priority to U.S. Provisional Application Ser. No. 63/150,632 filed Feb. 18, 2021, and this application is a continuation in part of PCT Application No PCT/US20/49638 filed Sep. 8, 2020 which claims priority to U.S. Provisional Application Ser. No. 62/898,312 filed Sep. 10, 2019, the content of which is incorporated herein in its entirety.

### STATEMENT OF GOVERNMENT SUPPORT

This invention was made with government support under R01 DC014547 awarded by the National Institutes of Health. The government has certain rights in the invention.

### FIELD

The disclosed subject matter relates to a microneedle, a device, a system and method for the precision delivery of drugs across membranes via perforations induced by microneedle arrays. More specifically, the subject matter relates to a micro-scale, ultra-sharp needles with precise geometries carry vital importance for overcoming various barriers, and have immediate applications for inner ear delivery and fluid sampling.

### BACKGROUND

Hearing loss is the most common sensory disturbance in humans affecting nearly 10% of the U.S. population. Other disorders of the ear include balance disturbance, Meniere's Disease, sudden sensorineural hearing loss and tinnitus. In order to treat these disorders, it is often desirable to administer therapeutic agents, e.g., medications or other medical fluids, into the middle and/or inner ear of a patient (see FIG. 1). These symptoms, either alone or in combination, can be reflective of underlying otologic disorders that necessitate specific medical or surgical intervention. Despite previous research and innovation, effective treatments for common inner ear illnesses such as sudden or progressive sensorineural hearing loss (SNHL) and Meniere's disease have remained particularly elusive, in part due to the anatomic inaccessibility of the cochlea. One known technique for administering such agents is intratympanic perfusion, as discussed in U.S. Pat. No. 7,840,260, the entirety of which is hereby incorporated by reference. Intratympanic delivery of drugs is typically accomplished by surgery involving making a small incision in the ear canal, i.e., anesthetized tympanic membrane (eardrum) and lifting the ear drum to create an access point to the middle ear. Once the access is available, the medical provider inserts a needle or catheter into the middle ear, infusing the drug in liquid form and allowing it to be absorbed into the inner ear by diffusion across the round window membrane (RWM).

Other methods have included placing an incision or implanted tube in the tympanic membrane and then having the patient self-dispense the drug into the external ear canal whereby it is intended to pass through the opening into the middle ear, and thence the inner ear.

These conventional techniques have many disadvantages. Many therapeutics are not capable of diffusing across the RWM due to their size or molecular weight. Further infectious debris can be carried into the middle ear from the external canal, with the risk of creating a middle ear infection, and passage of the liquid into the middle ear is inhibited by the surface tension of the liquid. Current methods of therapeutic delivery to the cochlea are inherently imprecise and can result in functional damage to the auditory and vestibular systems.

Protected by one of the hardest bones in body, the cochlea is a nearly impenetrable structure frustrating both bacteria and clinicians trying to gain access to it. Consequently, means for reliable delivery of agents into the inner ear for therapeutic purposes remains a formidable challenge. Were it not for its oval and round “windows”, delivery of therapeutic agents to the inner ear would always necessitate traumatic disruption of its bony walls with fearful consequences to hearing. Thus, the RWM is an attractive target for intracochlear delivery of drugs or biologic agents as it can avoid traumatic disruption of bony walls of the cochlea with fearful consequences to hearing. However, there is a need for a product that is capable of controlled penetration of the RWM to allow local delivery of therapeutic agent into the inner ear. Commercial products to locally deliver a drug proximally adjacent to the RWM solely relied on diffusion across the membrane for treatment. However, these commercial products have been largely abandoned as they have not dependably delivered material into the cochlea. Moreover, simple diffusion of drugs or agents across the RWM is limited by the type of material suitable for delivery, e.g., size of material to be delivered, difficulty with precise dosing, timing, and precision of delivery over time.

For example, transtympanic therapy with gentamicin and steroids is an important part of therapeutic armamentarium for the treatment of Meniere's disease. However, transtympanic therapy is associated with significant variability in clinical response and toxicity that is in large part related to the variable intracochlear bioavailability of the drug.

Delivery of a precise dose of medication across other anatomic barriers to the Central Nervous System (CNS) is also a serious challenge for clinicians of multiple specialties.

Thus, there remains a need for an apparatus and corresponding method that facilitate local delivery of therapeutic agents across anatomic membranes for reliable and predictable drug delivery without anatomic or functional damage.

### SUMMARY OF THE DISCLOSED SUBJECT MATTER

In one aspect, the disclosed subject matter is a solid or hollow metal microneedle intended for perforation of anatomic barriers such as the Round Window Membrane for the purpose of drug delivery or sampling of fluids.

The microneedle has a maximum diameter of about 20 microns, which is a critical size to enable the microneedle enables to penetrate the round window membrane of the inner ear to create a temporary, self-closing perforation. In some embodiments, the maximum diameter is about 10 microns.

The proximal portion of the microneedle may be hollow or solid and optionally configured with a taper along its length. The taper may be a gradual taper such as a gradual decrease in diameter along the length of the microneedle, or a stepped taper with abrupt changes in diameter that serve as reinforcing ribs or ledges. The distal portion comprises a taper to a pointed tip that facilitates penetration of an anatomic membrane. In some embodiments, there may be a narrow region of the microneedle at the junction between the distal portion and the proximal portion. For example, the microneedle may be configured wherein the proximal portion comprises a shaft and the distal end comprises a wide base and a narrow tip. The base of the distal portion may comprise one or more projections or barbs that engage the distal side of the membrane after penetration through the membrane and is held in place thereby. The barb(s) may provide the distal portion with a fishhook-like or arrowhead-like configuration.

Another aspect provides an array comprising one or a plurality of the microneedle described above. The microneedle array can be advanced through and penetrate an anatomic membrane, such as the round window membrane of the inner ear to create temporary, self-closing perforation(s). The temporary perforations allow access to the inner ear for local drug delivery of therapeutic agents.

The microneedles may be arranged in a regular pattern such as in an ordered array or disordered in a random pattern. In one embodiment, the micro needles are arranged in an array, for example a 10 by 10 array. The size of the array however may be dependent on the desired dosage of therapeutic agent. For example, the consistent delivery of therapeutic agent within the biodegradable distal portion by a 10 by 10 array provides a dosage of therapeutic agent that is four times the amount delivered by a 5 by 5 array, and so on.

In another aspect of the disclosed subject matter, a medical device capable of creating temporary perforations in the round window membrane of an inner ear is provided. The medical device includes a plurality of the microneedles described above. The plurality of microneedles is coupled to a base, which is configured to physically engage a driver device. Thus, both the medical device and the driver can be separate components that are engageable to each other to define a modular system.

In another embodiment a system for delivering therapeutic agent to the inner ear of a subject is provided which comprises an instrument for accessing the round window membrane; a plurality of microneedles, each microneedle having a diameter of about 10-20 microns with sufficient rigidity to perforate the round window membrane; and a driver, wherein the plurality of microneedles is coupled to the driver.

In another aspect, a method of delivering a therapeutic agent through an anatomic membrane is provided which comprises positioning at least one microneedle as described herein proximate the membrane wherein the microneedle is configured to penetrate the membrane; perforating the membrane; and dispensing a therapeutic agent at said perforation(s).

An embodiment provides for delivering a therapeutic agent into the cochlea comprising positioning at least one microneedle as described herein proximate the round window membrane wherein the microneedle is configured to penetrate the round window membrane; perforating the round window membrane; and dispensing a therapeutic agent at said perforation(s).

In another embodiment, the system may further include an indicator disposed along the system, such as a sensor, to indicate when the membrane is fully penetrated by the microneedles. For example and not limitation, a sensor may be included that is capable of sensing penetration into fluid. The sensing of penetration into fluid indicates that the membrane is fully penetrated.

In another embodiment, the system further includes an aspirating lumen within at least one microneedle that is connected to a suction device, e.g. sump. With respect to the aspirating lumen, fluid from the middle or inner ear can be aspirated before, during or after local delivery of therapeutic agent. The system and components can be disposable, single-use products.

Thus, described herein is a medical device and system for delivering a therapeutic agent into the cochlea comprising an instrument for accessing the round window membrane, at least one microneedle, the at least microneedle having sufficient rigidity to perforate the round window membrane, and a delivery mechanism for dispensing a therapeutic agent at said perforation(s).

In accordance with another aspect of the disclosed subject matter, a method of delivering a therapeutic agent into the cochlea is disclosed which comprises providing at least one microneedle on an instrument, positioning the at least one microneedle within the inner ear, perforating the round window membrane, and dispensing a therapeutic agent at said perforation(s).

The invention also relates to the manufacturing of solid or hollow metal microneedles and microneedle arrays. The method comprises:
(1) preparing a mold comprising at least one mold cavity on a conductive substrate by multiphoton lithography of a photoresist material;
(2) electrodepositing metal in the mold cavity to provide a microneedle; and
(3) removing the mold and conductive substrate from the microneedle.

In embodiments, the microneedle is solid, or the microneedle is hollow. In some embodiments, the mold comprises a plurality of mold cavities and an array comprising a plurality of microneedles is provided. In some embodiments, the method further comprises depositing a layer of conductive material on the surface of the mold cavity by physical vapor deposition (PVD), such as sputter deposition, cathodic arc deposition, electron beam heating, chemical vapor deposition (CVD) or atomic layer deposition (ALD) prior to electrodeposition.

In another aspect of the disclosed subject matter, a glassy carbon microneedle is provided. The glassy carbon microneedle is a pyrolyzed microneedle comprising a pyrolyzed body having a tip, base, and length therebetween. The pyrolyzed body comprises an atomic percentage of carbon of greater than 85%. For example, but not limitation, the atomic percentage of carbon is greater than 90%, or in some embodiments greater than 94%. The pyrolyzed body may also contain oxygen and silicon, but is substantially free of other atomic elements. For example, in some embodiments the body is nitrogen free. The body may be solid or hollow. The pyrolyzed microneedle is reproducible and scalable to manufacturing quantities with high yield.

The outer diameter of the pyrolyzed body is between 20 to 50 μm. The length (or height) of the pyrolyzed body is between about 100 to about 200 μm. The tip is can be conically shaped having a tip of radius of about 1 μm. In some embodiments, the pyrolyzed microneedle has Young's modulus of about 9 GPa a Weibull modulus of about 3, and/or a characteristic strength a of about 710 MPa.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 is a schematic representation of the ear anatomy.

FIG. 2A-2C are schematic representations of an exemplary method for preparing a mold suitable for preparing metal microneedles.

FIG. 3A-3C are schematic representations of another exemplary method for preparing a mold suitable for preparing metal microneedles.

FIG. 4A-4C are schematic representations of preparing microneedles using the mold prepared according to the embodiments.

FIG. 5A-5C are schematic representations of an embodiment of a method for preparing hollow microneedles.

FIG. 6 illustrates a microneedle array with 0.5 micrometer tip radii of curvature that can be prepared according to the exemplary methods described herein.

FIGS. 7A and 7B are schematic representations of an exemplary device having a plurality of microneedles in accordance with an exemplary embodiment of the disclosed subject matter.

FIG. 8 is a schematic representation of one embodiment of a microneedle having a central reservoir lumen.

FIG. 9 is a schematic representation of an exemplary device having a plurality of microneedles and coupled to a delivery device in accordance with one embodiment of the disclosed subject matter.

FIG. 10 is a schematic representation of the microneedle in accordance with the disclosed subject matter.

FIG. 11 is a cross-sectional view of the design of a needle mold containing a central circular current thief geometry with a diameter of 600 μm.

FIG. 12 (a) illustrates a computer generated rendering of microneedle design.

FIG. 12 (b) illustrates an optical micrograph of additively manufactured copper needle (with 100 μm shaft diameter) mounted on a 24 gauge stainless steel hollow blunt needle (with nominal 565 μm outside diameter).

FIG. 13 (a) illustrates a flat needle in accordance with another embodiment of the disclosed subject matter.

FIG. 13 (b) depicts a scanning electron micrograph of the flat-sided needle of FIG. 13(a).

FIG. 14 illustrates tip of a copper microneedle after cleaning.

FIG. 15 illustrates confocal micrograph of a round window membrane perforated with a metallic microneedle, 10× magnification.

FIGS. 16 (a) and (b) illustrate EBSD analysis showing copper microstructures of regions closer to the (a) tip of the needle, (b) base of the needle. Different shades depict individual grains.

FIG. 17 illustrates a scanning electron micrograph of a microneedle tip post perforation, showing no damage on the tip.

FIG. 18 is an optical micrograph of a gold-coated microneedle.

FIG. 19 illustrates SEM backscatter detection image showing small amounts of dark contamination on the surface, possibly carbon, but an otherwise conformal coating.

FIG. 20 depicts scanning Electron Microscopy of an electrochemically deposited needle tip, illustrating the imprints of voxel lines generated during the 2PP of the molds.

FIG. 21 illustrates a microneedle in accordance with a further embodiment, designed to mount on blunt stainless-steel hollow needle.

FIG. 22 illustrates a process flow for the fabrication of pyrolyzed microneedles.

FIG. 23(a) is an optical image of a 3D printed polymer microneedle;

FIG. 23(b) is a SEM image of the corresponding pyrolyzed microneedle of FIG. 23(a) mounting on Gauge 32 stainless steel syringe tip.

FIG. 23 (c) is a SEM image of the enlarged view on the pyrolyzed microneedle's tip of FIG. 23(a)-(b).

FIG. 24 illustrates the Raman spectroscopy analysis result for the pyrolyzed carbon microneedle.

FIG. 25(a) is a SEM image of a representative pyrolyzed micropillars array on a silicon substrate.

FIG. 25(b) is a SEM image of the enlarged tilted view of a micropillar (diameter=22 μm, height=70 μm).

FIG. 25(c) illustrates the discrete retardation spectrum in prony series of the material.

FIG. 25(d) is a comparison between fitting and experiment data.

FIG. 26(a) is a representative of the stress vs. strain curve to measure Young's modulus of the material.

FIG. 26(b) illustrates the measured Young's modulus values of six samples.

FIG. 27(a) is a representative of the stress vs. strain curve for compressive strength.

FIG. 27(b) is a Weibull probability plot for the derived glassy carbon material.

FIG. 28(a) is an optical image illustrating the in vitro perforation experiment setup.

FIG. 28(b) is a confocal image of a perforated RWM.

FIG. 28(c) is a SEM image of the microneedle's tip after perforation.

FIG. 28(d) is a force vs. time plot for a representative perforation experiment.

FIG. 28(e) is a box plot for measured maximum perforation forces.

### DETAILED DESCRIPTION OF EXEMPLARY EMBODIMENTS

The methods and systems presented herein relates to a system for treatment of the middle ear and/or inner ear disorders, and includes an apparatus and method for the compact, selectively controlled and metered introduction of a medical fluid, such as a drug, into the inner ear of a patient. Particularly, the presently disclosed subject matter is directed towards an apparatus having a plurality of microneedles for creating temporary perforations, i.e., perforations that close or heal within a week, in the round window membrane (RWM) which allow for reliable and predictable intracochlear delivery without permanent anatomic or functional damage. In some embodiments, the apparatus is a single microneedle.

Unless defined otherwise, all technical, mathematical and scientific terms used herein have the same meaning as commonly understood by one of ordinary skill in the art to which this disclosed subject matter belongs. Although any methods and materials similar or equivalent to those described herein can also be used in the practice or testing of the present disclosed subject matter, this disclosure may specifically mention certain exemplary methods and materials.

As used herein and in the appended claims, the singular forms “a,” “an,” and “the” include plural referents unless the context clearly dictates otherwise. Where a range of values is provided, it is understood that each intervening value between the upper and lower limit of that range and any other stated or intervening value in that stated range, is encompassed within the disclosed subject matter.

In one aspect, an ultra-sharp solid metal microneedle with complex architecture is disclosed. Additive manufacturing methods are employed to fabricate the microneedles by electrochemically depositing copper into polymeric molds fabricated via 2PP lithography. The resulting microneedles made by this process are fully metallic microneedles having superior precision. The tip of the microneedles have a radius of curvature as small as 1.5 μm and shaft diameter of 100 μm. Similar-sized polymeric microneedles are able to perforate the round window membrane with no long-term hearing loss. Healing may progress between 24 h to 48 h and the perforation can fully close within 1 week. In some embodiments, copper microneedles can be coated with a nickel film followed by a gold film, thus rendering the microneedle surfaces to be biocompatible.

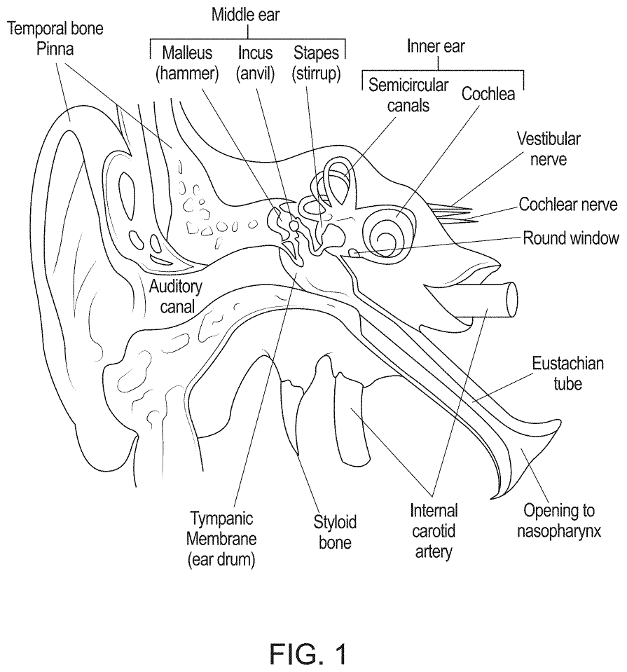

As shown in FIG. 1, the anatomy of the ear includes a middle ear 101 comprising the hammer 101a, anvil 101b, and stirrup bones 101c, and an inner ear 102 comprising the semicircular canals 102a and cochlea 102b. The middle ear 101 and inner ear 102 have barriers to entry and are separated from auditory canal 103 by the tympanic membrane 104 or ear drum. Moreover, the inner ear 102 is further protected from entry by its almost impenetrable structure. The round window membrane (“RWM”) (secondary tympanic membrane) 105 disposed at the inner ear 102 provides an avenue to permit local delivery of therapeutic agents directly to the inner ear.

### Round Window Membrane

The RWM 105 is a three layered structure designed to protect the inner ear from middle ear pathology and facilitate active transport. There is an outer epithelial layer that faces the middle ear, a central connective tissue layer, and an inner epithelial layer interfacing with the scala tympani. The most prominent feature of the outer epithelial layer is the extensive interdigitations and tight junctions of its cells; in addition, there is also a continuous basement membrane layer. This architecture with tight junctions and a continuous basement membrane functions as a defensive shield designed to protect the inner ear from middle ear infections. The connective tissue core contains fibroblasts, collagen, and elastic fibers, and houses blood and lymph vessels. The connective tissue is divided roughly into thirds differing in fiber type and density thus essentially establishing a gradient. This layer is responsible for providing compliance to the RWM. Finally, there is a discontinuous inner epithelial layer that bathes in the perilymph of the scala tympani. As previously noted, conventional transtympanic delivery is limited as it relies on the ability of particles to diffuse or be actively transported across this three-layered membrane.

A large range of materials are able to cross the RWM, including various antimicrobials, steroids, anesthetics, tracers, albumin, horseradish peroxidase, latex spheres, germicidal solutions, water, ions, and macromolecules (including bacterial toxins) as long as the materials are suitable for simple diffusion transport. Several factors contribute to the RWM permeability, including size, charge, liposolubility, the morphology of the compound, and the thickness of the RWM. Size has proven to be a factor in permeability, as 1 μm microspheres cross the RWM, but 3 μm microspheres cannot. Furthermore, substances with a molecular weight of less than 1000 kDa diffuse across the RWM fairly rapidly, whereas substances over 1000 kDa require pinocytosis to cross the RWM. Charge of the molecule can also impact its ability to traverse the RWM; for example, it has been noted that cationic ferratin crosses the RWM, but anionic ferratin does not. Finally, increased thickness of the RWM will decrease permeability of substances. While the average thickness of the human RWM is between 70 and 80 μm, this thickness can double in inflammatory conditions. RWM permeability can be altered with the use of exogenous adjuvants such as histamine (for its vasodilatory effects), hyaluronic acid (for its proposed osmotic effect), and dimethylsulfoxide (for its ability to increase medication solubility in perilymph); however, their clinical applications are limited. Consequently, a major limitation of conventional transtympanic delivery method that takes advantage of this natural permeability of the RWM is the great variability in intracochlear delivery of the therapeutic agent; this leads to variation in clinical response and toxicity. Furthermore, many therapeutics cannot be delivered due to the molecular size and weight. The systems and methods described below provide a solution to the problem of local drug delivery to the inner ear, and is not limited by factors required for simple diffusion.

### Microneedles to Create Micro-Perforation of RWM.

To overcome the limitations of diffusion based delivery across RWM, the present embodiments create controlled micro-perforations through the RWM with a single or plurality of microneedles that: 1) improves the diffusive permeability of RWM dramatically and controllably; 2) minimizes the damage to the RWM cellular architecture so that RWM heals itself by self-closing in a period of time; 3) prevents the convective perilymph leak by the cerebrospinal fluid (CSF) pressure and prevent unintended disruption of endocochlear pressure fluctuation, and 4) locally delivers drugs or compounds that cannot diffuse across the RWM.

Polymeric ultra-sharp needles can be manufactured utilizing recent advances in 2-photon lithography at the scale necessary to deliver therapeutic materials across the dermis, but these micro-needles do not have the precision nor the capability to deliver therapeutic materials across the round window membrane by creating temporary perforations that self-close within a week, and preferably 24 to 48 hours and/without damaging the to the RWM.

The techniques described herein allow for the fabrication of metal microneedles, which differ from the micro-manufactured needles that have been produced thus far with other technologies. The microneedles of described and claimed herein can be manufactured with increased design freedom than other metal needles.

The fully metallic microneedle provides stronger needles that retain their sharpness after undergoing larger amounts of stress compared to plastic replicas, since metals typically have higher yield stresses. Furthermore, grain size of the electrodeposited metal can be controlled using various means, which gives the manufacturer control over the mechanical properties of the needles. The needles can thus be harder and more brittle at certain sections and softer and more ductile in others.

Ultra-high precision 3D molds can be made via 2-photon lithography. Two photon lithography can be used to manufacture molds for making thermoplastic microneedle arrays for drug delivery and fluid sampling across the anatomic membranes the ear, eye and the CNS such as the RWM.

Manufacturing precision microneedle or microneedle array molds using 2-photon lithography allows for each of the following novel improvements to existing needle technology, such as direct manufacturing of slanted or curved needles or needles with complex geometries or base structures for difficult-to-reach anatomic areas; injection molding of biodegradable microneedle arrays with barbed or fishhook-style fasteners at the base of individual needles, allowing for securely embedding the array within tissue membranes for days/weeks following the implantation and/or hollow or solid needles that detach upon insertion, break down and release contents; injection molding of internal reservoirs, either within the body of the needle or the base, to contain a precise amount of pharmaceutical, molecular or cellular therapeutic material and releasing that material into closed anatomical spaces in a controlled manner; and direct manufacturing of microscopic hollow needles for the controlled delivery of pharmaceutical, molecular or cellular therapeutic materials contained within microscopic capsules.

The process involves micromolds fabricated by 3D stereolithography directly from CAD drawings, which are then used to provide shaped metal microneedles using eletrodeposition techniques. High precision 3D molds can be manufactured via two-photon lithography. The fabrication method starts with manufacturing of the molds on a conductive substrate. Microneedles having a 1.0 micron tip and a 0.5 micrometer radius of curvature tip can be manufactured. Using the same order of magnitude feature sizes, the molds are made in the negative image of the desired needles by curing photoresist using two-photon lithography. The uncured photoresist is then stripped away by means of chemical treatment to leave cavities or voids in the mold. The substrate would then be left bare only at locations where the tips of the microneedles will be.

The substrate needs to have a conductive surface to enable electrodeposition of metal. Every part of the conductive surface, except for the bare parts inside the patterned molds will need to be masked before being submerged into the electrolyte for electrodeposition. The mold-substrate assembly would then be submerged into an electrolyte and electrochemical deposition of metal would occur, filling the cavities or voids inside the molds, taking the shape prescribed by the voids.

After submersion, electrodeposition is conducted by flowing current or applying voltage to the system, growing the needles, tip-first, into the voids of the molds. The current density may be controlled by predicting the necessary current through mathematical models or keeping a constant voltage throughout the process.

After the molds are filled with metal, the molds can be stripped away by means of heat treatment or chemical treatment. The metal needles may be released as a final step by electropolishing or etching the underlying layer of material.

Full metal needles will be manufactured in the shape of the voids inside the 3D lithographed molds, and this process in itself provides design freedom that is well suited and sometimes critical for a variety of applications. The needles can be slanted, curved or can take a shape necessary to conform to the necessities of the application in question.

In some embodiments, the microneedle(s) may be prepared in an array comprising a plurality of microneedles joined by a field comprising the same metal of the microneedles. Preferably, the microneedle array can be prepared in a single process wherein the microneedles are prepared in the array as a unitary shaped article.

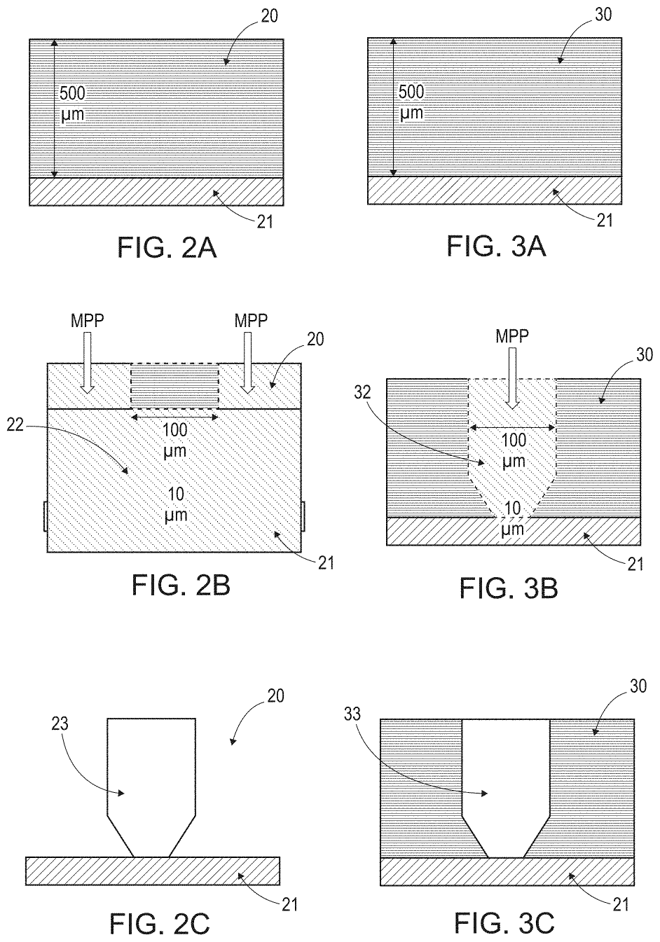

FIG. 2A through FIG. 2C schematically illustrate a method of generating a mold for preparing an array having a plurality of microneedles. For simplicity, the Figures illustrate an array with a single microneedle shown in cross-section. The microneedle is 500 μm in length with a main shaft diameter of 100 μm tapering to a tip diameter of 10 μm (shown not to scale in the Figures). A layer of mold precursor compound 20 is formed on a substrate 21 so as to provide a mold precursor having a cross section as shown in FIG. 2A. The mold precursor compound 20 can be a negative photoresist. In some instances, the photoresist includes or consists of a photopolymer. Photopolymers change their chemical properties when exposed to light or light of certain wavelengths. In some instances, the photopolymer changes its solubility in a lithography developer in response to exposure of the photopolymer to light of a particular wavelength or range of wavelengths. For instance, a suitable photopolymer can polymerize and/or crosslink in response to exposure of the photopolymer to the light. An example of a suitable photopolymer includes, but is not limited to, IP-Dip 780 photoresist available from Nanoscribe, Inc. Suitable materials for the substrates 21, include, but are not limited to, metals such as aluminum. The substrate may also comprise a nonconductive backer, such as glass or polymer, coated with a conductive surface. For example, the substrate may comprise a glass slide with a coating of indium titanium oxide (ITO) sputtered onto the glass slide to provide an electrically conducting substrate.

A mold cavity 23 of the desired shape is formed in the mold precursor 20 of FIG. 2A to form a mold as shown in FIG. 2B and FIG. 2C. A suitable method for forming the mold 23 in the mold precursor 20 includes, but is not limited to, multiphoton photolithography such as two-photon lithography. Other names for multiphoton photolithography include direct laser writing and direct laser lithography. In multiphoton photolithography, the mold precursor 10 is transparent or substantially transparent to the wavelength of a light source so as to suppress single photon absorption relative to multiphoton absorption. The multiphoton absorption can cause the desired chemical change of the mold precursor 20. For instance, when the mold precursor 20 includes or consists of a photopolymer, the multiphoton absorption can cause polymerization of the photopolymer and/or cross-linking of the photopolymer. When the photopolymer is IP-Dip 780 photoresist, the multiphoton absorption can cross-link the polymer.

The mold precursor 20 is subjected to multiphoton photolithography, signified as MPP in FIG. 2B, in a pattern that provides a region of cross-linked polymer in the mold precursor 20 except in the region(s) planned to be in the desired shape of a cavity precursor 22. As will become evident below, the mold cavity 23 will be filled by one or more materials that serve as the composition of the microneedle (and array). Accordingly, the arrangement of the cavity precursors 22 and cavities 23 is the same as or approximates the desired arrangement for the microneedles in the resulting array and the mold cavities 23 have the same dimensions as are set forth for the microneedles.

In some instances, the light source used for multiphoton absorption is configured to have a focal point. In some instances, the light intensity requirements needed for multiphoton absorption cause the desired chemical change to occur at the focal point or focal volume of the light source without substantially occurring outside of the focal volume. By controlling the location of the focal point or focal volume of the light source within the mold precursor 20, the location of the chemical change within the mold precursor 20 can be controlled. For instance, the relative positions of the device precursor and light source can be changed such that the focal point or focal volume of the light source effectively scans around the desired locations of the mold cavities 23 within the mold precursor 20. Since the desired chemical change is localized relative to the focal point or focal volume of the light source, the use of multiphoton absorption permits the array features to be formed with the above dimensions. Further, since the desired chemical change is localized to the focal point, the chemical change does not substantially occur between the light source and the focal point. Accordingly, a trace of the chemical change does not occur between the target location of the cavity 23 and the light source. As a result, features can be formed centrally within the mold precursor 20 without the feature extending to the perimeter of the mold precursor 20. The ability to form features centrally within the mold precursor 20 permits the formation of mold cavities in nearly any configuration.

The non-crosslinked mold precursor in the cavity precursor 22 can be removed from the mold precursor of FIG. 2B to reveal the mold cavity 23 of FIG. 2C. For instance, when the mold precursor 20 comprises a negative photoresist, the mold precursor 20 can be removed with a suitable developer to reveal the mold cavity 23. A suitable developer for IP-Dip 780 comprises propylene glycol monomethyl ether acetate, PGMEA.

In a specific embodiment, the molds for the microneedles may be fabricated by 3D laser lithography using the Photonic Professional GT system (Nanoscribe GmbH, Karlsruhe, Germany). The direct laser writing (DLW) technique also known as two-photon polymerization (TPP) or 3D laser lithography is a nonlinear optical process based on two-photon absorption (TPA) theory. The Nanoscribe system is equipped with a pulsed erbium-doped femtosecond (frequency-doubled) fiber laser source with a center wavelength of 780 nm for the exposure of the photoresist. At the pulse length of 100-200 femtosecond, the laser power ranges between 50-150 mW. For fabrication of microneedles CAD models may be generated by SolidWorks software (Dassault Systems SolidWorks Corporation, Concord, N.H., USA) in stereolithography (STL) file format and imported to the software package Describe (Nanoscribe GmbH, Germany) for scripting of writing parameters. The laser beam is focused into the negative-tone photoresist, IP-S (Nanoscribe GmbH, Karlsruhe, Germany), using a Dip-in laser lithography (DiLL) objective with ×25 magnifications and NA=0.8.

In this process, the objective lens is directly dipped into the liquid and uncured photoresist acts as both photosensitive and immersion medium in an inverted fabrication manner. The refractive index of the photoresist defines the focal intensity distribution. For the DiLL process, the objective working distance does not limit the height of the sample; therefore, structures with micrometer to millimeter heights can be fabricated. A drop of resist is cast on the substrate; IP-S exhibits good adhesion on the substrate, and is loaded onto the system. Microneedle arrays are written in galvo scan mode (XY) and piezo Z offsetting mode. The arrays may be split into blocks of about 200-400 μm×200-400 μm×10-30 μm (XYZ), within the working range of the galvo scan mode. Blocks can be stitched together to create larger arrays. Depending on the design, the laser power can be 50-150 mW, with scan speed of 5-10 cm s⁻¹, with minimum and maximum slicing distance 0.1 and 0.5 μm. After exposure, the structures are developed in propylene glycol monomethyl ether acetate (PGMEA) bath for 10-60 minutes plus a 2-10 minute isopropyl alcohol (IPA) rinse followed by 20 min flood exposure through a UV light source with 16 mW cm-2 intensity to further crosslink the photosensitive material.

FIG. 3A through FIG. 3C illustrate another embodiment of method for generating a mold. A mold precursor 30 is formed on a substrate 21 to provide a mold precursor having a cross section as shown in FIG. 3A. The mold precursor 30 can be a positive photoresist. In some instances, the photoresist includes or consists of a photopolymer. In some instances, the photopolymer changes its solubility in a lithography developer in response to exposure of the photopolymer to light of a particular wavelength or range of wavelengths. For instance, a suitable photopolymer can polymerize and/or cross link in response to exposure of the photopolymer to the light. An example of a suitable photopolymer includes, but is not limited to, AZ4620 available from Microchem Corp. located in Newton, Mass.

Mold cavity precursors 32 are formed in the mold precursor 30 of FIG. 3A by multiphoton photolithography similarly to that described above except that the laser is focused to be absorbed in the region(s) where the mold cavity 33 is to be created. In this instance, the crosslinked polymer becomes more soluble in a suitable developer than the non-crosslinked polymer. Treatment with the developer results in the removal of the crosslinked polymer from the mold cavity 33. A suitable developer for AZ6240 is a solution of potassium borates, commercially available as AZ400k from AZ Electronic Materials, Somerville, N.J.

The mold cavity precursors 32 can be removed by developing and/or etching. For instance, when the mold precursor 30 is AZ4620, the mold cavity precursors 32 can be removed by developing and oxygen plasma etching. The removal of the mold cavity precursors 32 leaves mold voids 33 in the mold precursor 30. As will become evident below, the remaining mold precursor 30 acts as a template for the microneedle array deposition.

The use of either positive or negative photoresists in the mold forming steps may be determined by the particular design of the microarray desired. For example, a positive photoresist may be more desirable if the volume of mold cavities is small relative to the volume of remaining mold precursor in the mold template. Other factors governing choice of photoresist may include the resolution needed for the details of the particular microneedle design, the strength of the mold template during elecrodeposition, and/or the ease of removal of the mold material after the elecrodeposition.

In either instance, the mold cavities 23 or 33 may be further treated with, for example, oxygen plasma treatment or photoetching to refine the resolution of the cavities. Oxygen plasma treatment increases the free energy of the surface by creating hydrophilic, oxygen-containing groups such as carbonyl and carboxyl esters on the surface. Oxygen plasma treatment may be performed on the thermoplastic microneedle mold arrays using an oxygen plasma etcher (PE-250 Plasma etcher, Denton vacuum, USA) with 50 W RF power and 340 mTorr pressure for 20 min.

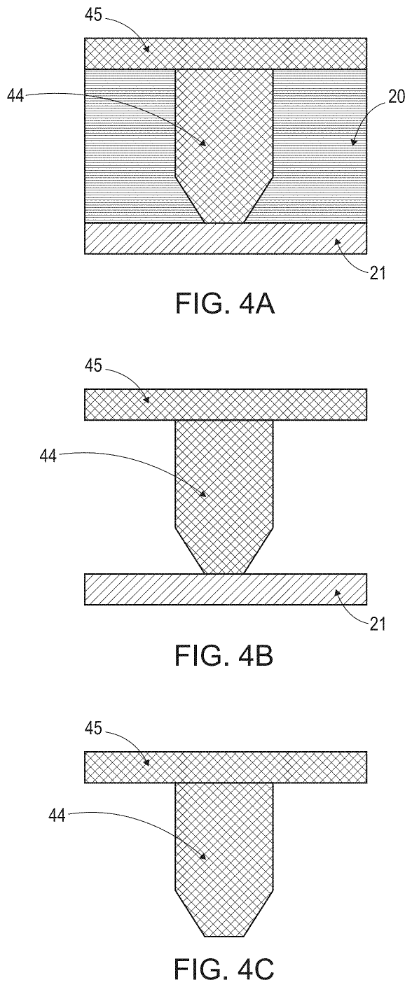

The mold cavities (or voids) 23 or 33 in the molds are fully or partially filled with metal to provide the microneedles. Suitable methods for filling the mold cavities include, but are not limited to, electroplating or electrodeposition. As described above, when filling the mold cavities with a metal, the substrate is electrically conducting and the substrate can be used to electroplate the interiors of the mold cavities. When the mold is treated in a suitable electrochemical cell with an appropriate electrolyte, atomic metal is deposited on the surface of the exposed conducting substrate. As metal is deposited in the cavity in contact with the electrically conducting substrate, it also becomes conductive, allowing additional metal to be plated thereon. In this way, the mold cavities may be filled from the microneedle distal end or tip to the proximal end. A schematic illustration of a filled mold cavity in the mold is shown in FIG. 4A, showing a solid microneedle 44 filling the mold cavity. In some embodiments, electroplating is continued after the mold cavities are filled to form a wider base at the proximal end of the microneedle. Continued electrodeposition results in a field of metal 45 linking the proximal ends (bases) of the microneedles, fixing the microneedles in the desired locations in the array. Example metals for the electrodeposition may include nickel, silver, titanium or copper, preferably copper.

Other suitable methods of filling the cavities include, but are not limited to, deposition and/or growth techniques such as chemical vapor deposition (CVD) or atomic layer deposition (ALD). These techniques are particularly useful when preparing microneedles with layers or shells comprising different materials. Since atomic layer deposition (ALD) can provide a coating with a thickness at the angstrom level, atomic layer deposition (ALD) is an example of a method that is suitable for preparing layers on the mold cavities having very small thickness levels. Atomic layer deposition (ALD) typically includes sequentially reacting different gas phase precursors with the surface of the mold cavities in a self-limiting chemical process. In some instances, the process is repeated in order to provide the shell layer with the desired thickness. Different shell layers can be formed using different deposition techniques or using the same deposition technique.

The remaining mold precursor is removed from the mold to leave microneedle 44 and array 45 attached to the substrate 21 as shown in FIG. 4B. The mold precursor may be removed by soaking in a suitable solvent such as N-methylpyrrolidone.

The substrate 21 is removed after fabrication of the microneedle and/or microneedle array. Suitable methods for removing the substrate 42 include, but are not limited to, etching and mechanical methods such as polishing. In some instances, the proximal end of the microneedle 44 or the upper surface of the array 45 may be bonded or attached to a second substrate (not shown) before removal of the substrate 42. Additionally or alternately, the proximal ends of the microneedles 44 can optionally be encapsulated in an encapsulating material that fills in the spans the gap between the microneedles in the array. Suitable encapsulating materials include, but are not limited to, polymers, or epoxies. In this way, solid microneedles may be prepared, as shown in FIG. 4C.

In some embodiments, the steps of removal of mold compound and removal of the substrate may be conducted in a different order. For example, substrate removal may be conducted prior to removal of the mold compound. In other embodiments, partial removal of the mold compound may be conducted, followed by substrate removal followed by removal of the remaining mold compound. The order of steps of removal of mold compound and substrate may be dependent on the particular design of the microneedle and/or array and any post-fabrication processing contemplated.

Hollow needles may be prepared similarly, except a column or shaft of mold material 20 or 30 surrounded by a ring of exposed substrate 21 within the microneedle mold cavity 22 or 32 is designed into the mold drawing process. Electroplating as described above fills the cavity surrounding the column or shaft with metal. Removal of the mold compound provides a microneedle comprising a central lumen.

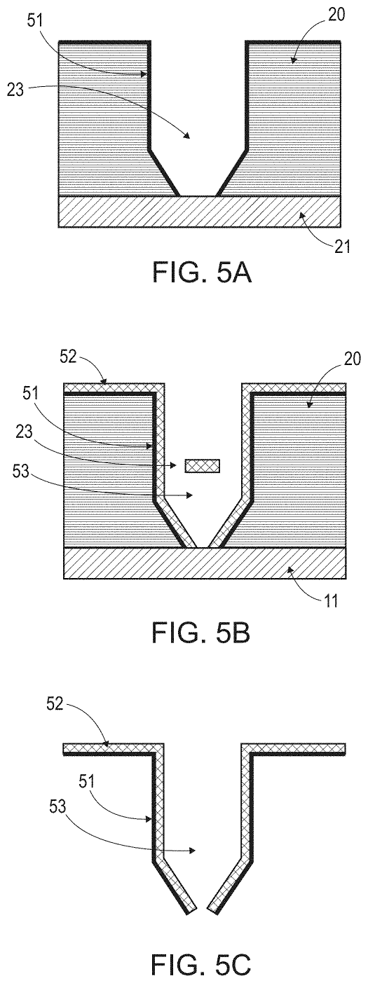

An alternative method for preparing hollow needles is shown schematically in FIGS. 5A to 5C. In this method, a coating of an electrically conductive material 51 is applied to the interior surface of the mold cavity 23 and optionally to at least a portion of the upper surface of the mold compound 20 as shown in FIG. 5A. In this way, the entire interior surface of the mold cavity is made conductive. Examples of conductive materials include metals such as copper, or compounds such as indium titanium oxide (ITO), titanium nitride (TiN), sometimes known as tinite, titanium carbon nitride (TiCN), titanium aluminum nitride (TiAlN or AlTiN), and titanium aluminum carbon nitride. These materials can be applied by physical vapor deposition (PVD, usually sputter deposition, cathodic arc deposition, electron beam heating, chemical vapor deposition (CVD) or atomic layer deposition (ALD). For example, copper can be applied using RF magnetron sputtering. A notable material is titanium nitride, an extremely hard ceramic material, often used as a coating to improve a substrate's surface properties. TiN is non-toxic, meets FDA guidelines and has seen use in medical devices such as scalpel blades where sharpness and edge retention are important. Titanium nitride may be applied by ALD. In some embodiments of this method, the substrate 21 need not be conductive, since it will also be coated with the conductive material when it is applied to the mold cavity 23.

In a second step, electrodeposition is conducted as described previously. However, instead of a small region of conductive material at the tip of the microneedle cavity due to the conductive substrate, the entire interior surface of the mold cavity is conductive, and electrodeposition results in a layer of deposited metal 52 over the mold cavity coating 51. It is not necessary to fill the entire cavity 23 with deposited metal, so a central lumen 53 remains within the mold cavity 23. Removal of the mold material 20 and the substrate 21 provides a hollow microneedle as shown in FIG. 5C. The resulting microneedle comprises two layers, with a coating or shell layer 51 on the outside of the main metal layer 52. This method may also be used to prepare solid microneedles if electrodeposition is conducted until the entire mold cavity 23 is filled.

The microneedle or array can be further treated after mold removal to provide a finished product by for example, polishing, etching and/or coating. For example, the exterior of the microneedle can be coated with a thin layer of TiN by ALD. The exterior of the needle may also be coated with a therapeutic agent, optionally encapsulated by a biodegradable polymer for delivery through a membrane.

The above methods of microneedle fabrication allow features to be formed centrally within the mold precursors 20 or 30. This ability allows the microneedles and/or arrays to be formed with more complex features. For instance, all or a portion of the microneedles in an array can be curved. These methods also permit a high level of resolution even at the nanometer and micron level dimensions disclosed above. As a result, the arrays can include more sophisticated construction features. For instance, the microneedles need not all have the same dimensions. Accordingly, in some instances, a first portion of the microneedles have different cross sectional dimensions than a second portion of the microneedles. For instance, the first portion of the microneedles may be solid or can have a thicker shell and/or core than a second portion of the microneedles. Alternately or additionally, the first portion of the microneedles can have larger cross sectional dimensions than a second portion of the microneedles. Accordingly, microneedles that will experience higher forces during use of the microneedle array can be designed to tolerate higher loads than other microneedles.

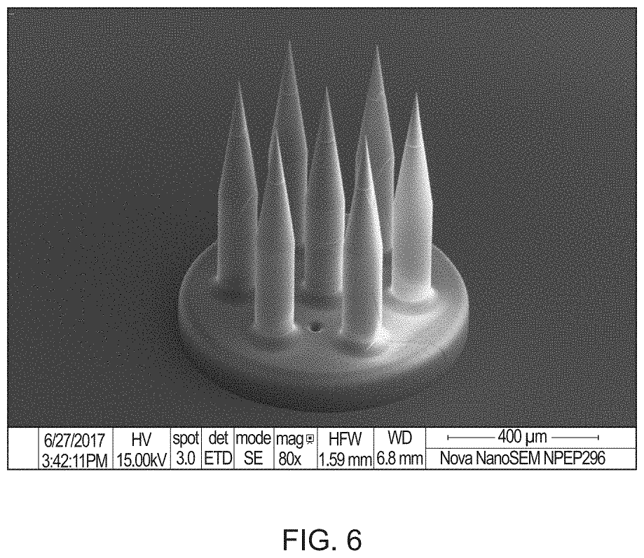

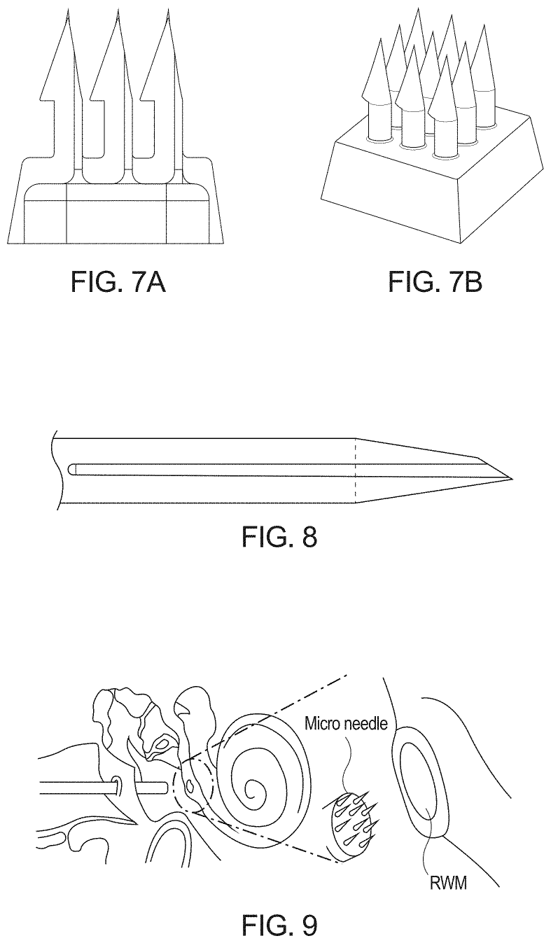

The microneedle arrays, in accordance with some embodiments, are shown in FIGS. 6, 7A, 7B and 8. FIG. 6 shows a microneedle array with 0.5 micrometer tip radii of curvature that can be prepared according to the methods described herein. The cross-section of the microneedles in the array is similar to that illustrated schematically in FIGS. 3A-5C. FIG. 7A shows a cross-section schematic of a microneedle array wherein the microneedles has a longitudinal body having a proximal portion, distal portion and a length therebetween. The proximal portion has a shaft having a length, and the distal end of the body tip. Proximal to the tip is a barb that has a width that extends laterally from the shaft. Accordingly, the barb extends laterally beyond the shaft. The barb is configured to engage the membrane after penetration and allow for such a device to be securely embedded in tissue membranes. The microneedle body also includes an open channels disposed at least within the shaft. In some embodiments, the open channel extends to a distal portion of the microneedle body. In some embodiments, the open channel is disposed along one side of the microneedle, i.e., is off-center from the central tip of the microneedle body. The open channel is configured for conducting fluid along the microneedle within the shaft to the exterior of the needle for delivery of therapeutic material to a patient. Thus, opening of the open channel for delivery may be offset and proximate the tip. FIG. 7B shows a perspective view of a 3 by 3 array of microneedles similar to those shown in FIG. 7A. These images also depict an open channel for therapeutic materials within the base of the array. FIG. 8 shows a microneedle with a central open channel having an opening for delivery of therapeutic material at the central axis of the tip. In this embodiment, the tip has a slanted configuration.

In accordance with an aspect of the disclosed subject matter, a device capable of locally delivering a therapeutic agent into the inner ear or cochlea is provided. The device includes a plurality of microneedles as described herein configured to controllably penetrate (to a desired depth) the RWM to create temporary access to the inner ear through temporary perforations. The plurality of microneedles may have a regular or ordered arrangement such as in an array, or have an irregular or random arrangement, if so desired.

The microneedles are suitably sized to create temporary perforations in the RWM without tearing or ripping the RWM. The term “controlled penetration” or “controlled perforation” means that the opening created by the microneedle has substantially regular or smooth edges, as opposed to a “tear” or “rip” which is to pull apart in a way that leaves ragged or irregular edges. The aperture created upon insertion of the microneedle does not expand or distort, but instead retains a shape and size which corresponds to the shape and size of the microneedle which created the aperture. The term “temporary perforations” means that the openings created by the microneedles self closes without the need for a wound closure procedure. In this regard, in one embodiment the microneedles have a diameter of about 10 micron. As discussed in further detail herein, it has been found that the size of the microneedle is important to create perforations or openings in the RWM that self-close fully within one week. In some embodiments, the perforations are self-close within 48 hours, or 24 hours from the perforation. The creation of temporary perforations in the RWM allows for reliable and predictable intracochlear drug delivery without permanent anatomic or functional damage to the ear.

A preferred microneedle array has an adequate safety margin to avoid failure while penetrating the RWM. The primary cause of a failure is buckling of needles. The safety margin is defined as the ratio of the force that that causes buckling of the needle array to the force required to penetrate the RWM.

A reliability estimate of the microneedles that compares the buckling force of a nanoindenter tip to the force at perforation, the ratio of which is the safety factor, FS. These results suggest that by reducing the tip diameter below 1 μm, the rupture force can be reduced down to 10 mN. Thus, a 10 μm and 20 μm radius needle will have FS of 2 and 36 respectively. The buckling force is calculated based upon modeling the actual geometry of the indenter tips fabricated and employed in the experiments. The pertinent boundary conditions are to fix one side against displacement and to allow the other end freedom to displace and rotate. Then a buckling analysis is performed to determine when the nano-needles are expected to fail due to lateral motion (either buckling or bending).

The force and displacement indicate that to produce a 20 μm diameter hole the needle should withstand an axial force of at least 10 mN. Also, a 100 μm length is necessary to ensure the substrate of the microneedle does not touch the RWM. To maintain a FS of 10 against buckling failure, a 10 μm diameter, 100 μm long Si column with tip size of 2 μm is used.

For purposes of illustration and not limitation, the microneedle of the present disclosure can be formed with a 0.5 μm tip, and a 20 μm diameter shaft which is 100 μm in length. Such a microneedle exhibits a 0.4. to 5 mN rupture force, a buckling load of 160 mN, with a safety margin of greater than 30. The microneedle can be formed with a gradual or stepped taper at the distal tip. The stepped taper configuration results in abrupt changed in diameter which can serve as structural reinforcing ridges for withstanding greater insertion loads without buckling or deforming. Additionally, or alternatively, the desired strength characteristics of the proximal portion of the microneedle can be achieved by selection of the material properties (e.g. tungsten vs. silicon).

In embodiments that employ a solid microneedle construction, the tip of the proximal portion of the microneedle(s) can be coated with a biodegradable material comprising a therapeutic agent material to form the distal portion and permit local delivery of the therapeutic agent into the inner ear. The coating may be configured so that it is releasably adhered to the tip of the proximal end and is held in place by pressure of the microneedle tip against the membrane while the microneedle is driven forward through the membrane. Once the membrane is penetrated, the distal portion engages the membrane and is pulled from the proximal portion as it is retracted. The driver may be configured to tilt the array after insertion of the distal portion and provide a shearing or prying force to facilitate cleavage of the distal portion from the proximal portion.

The microneedle arrays disclosed herein are designed for painless transmembrane administration of drugs, which can be delivered through perforations in an anatomic membrane. The size of the needle can be varied greatly depending on the tissue and the material to be injected.

In some embodiments, each microneedle can be formed with a uniform geometry such that each corresponding perforation is a uniform and constant depth. Additionally, or alternatively, select microneedles can be formed with differing geometries to provide a non-uniform or patterned perforation design. Furthermore, a greater concentration of microneedles can be provided at one portion of the RWM than another to provide the operator with greater flexibility and customization for different patients. Moreover, the microneedles can be formed with differing lengths which coincide or map to the contour of the RWM so as to ensure a uniform depth of insertion into the RWM across its varying or non-planar (i.e. “saddle point”) shape.

The array of microneedles can be mounted onto a surgical instrument (e.g. catheter) that allows access to the RWM either via the tympanic membrane or via the mastoid process. Once the microneedles are positioned proximate the RWM, the driver can operate to insert the microneedles into the RWM to create the perforations to the desired depth. After penetration, the distal portion will remain in the RWM and serve as a reservoir to deliver therapeutics at a controllable rate into the RWM, or distal to the RWM as desired. The proximal portion of the microneedles can be retracted from the perforations formed in the RWM.

The plurality of microneedles can be a component device that is configured to engage a surgical instrument for introduction into the ear, such as a driver, introducer, catheter, or other device. In this regard, the device includes a base and a plurality of microneedles. The base is adapted to mount onto a surgical instrument that allows access to the RWM either via the tympanic membrane or via the mastoid process. In this regard, the base can include threads to screw onto the surgical instrument. However, other structures for physical coupling to the surgical instrument can be employed as would be known to one of skill in the art, such as clips, snap-on friction fit engagement, and the like. An exemplary embodiment of the medical device is depicted schematically in FIG. 9 that illustrates a circular array positioned proximate to the RWM. Other arrays are also contemplated, such as polygonal or oval.

The surgical instrument can be configured for pediatric indication or adult indication. For example, the length and diameter of the surgical instrument can be smaller for use for pediatric treatment.

In another aspect, the subject matter provides an apparatus including the plurality of microneedles and driver formed as a unitary or non-separable device which can be disposable or reusable.

In another embodiment, the system or apparatus further includes an indicator to signal full penetration through the RWM. In this regard, the system or device may include a sensor to sense air, tissue, and/or fluid. Once the sensor senses fluid the sensor communicates with the indicator to signal full penetration through the RWM.

In yet another aspect, the system or apparatus may include an aspiration lumen and aspirator device. In this regard, the aspirator can aspirate fluid from the middle or inner ear, and deliver drugs locally to the middle or inner ear.

While the work described herein focuses on accessing the cochlea, the technology can be translated to other anatomic barriers and enclosed spaces in the eye and central nervous system. Biodegradable ultra-sharp microneedles could be used to deliver therapeutic materials across the meninges into the brain and spinal cord, across the sclera into the eye and across the nerve sheath into peripheral nerves. Controlled therapeutic delivery without functional damage to these anatomic targets remains a challenge. The reservoirs used to house therapeutic materials for delivery can be modified for various pharmaceutical, molecular or cellular therapeutic agents depending on the clinical need.

The microneedles can also be utilized as electrodes for stimulation of specific locations within the body.

### EXAMPLES

The following examples are exemplary and are not meant to be limiting.

### Example 1

Solid microneedles may be prepared as follows. The method of FIG. 3A through FIG. 3C is used to generate a mold. A 60 nm thick layer of indium titanium oxide (ITO) is sputtered onto a 170 μm-thick glass slide to provide an electrically conducting substrate. A positive photoresist of AZ4620 (Microchem) is used as a mold precursor. The mold precursor is spun onto the substrate with a thickness of 500 μm and soft-baked for 3 minutes at 110° C. Direct laser writing (DLW) using two-photon lithography forms the mold cavities within the mold precursor. The direct laser writing (DLW) is performed with a 780 nm wavelength laser. The laser-treated regions in the mold precursor are removed from the non-treated mold precursor to leave mold voids in the mold precursor. The mold members are removed by developing the mold precursor in 1:4 diluted AZ400 (Microchem) for eight minutes followed by exposure to oxygen plasma at 100 W and 300 sccm for two minutes. The resulting mold voids are opened all the way down to the substrate to provide a conductive starting point for the electrodeposition. Potentiostatic electrodeposition is then used in a miniature three-electrode electrochemical cell in order to place the material for the microneedles in the voids. In one instance, the mold voids are filled with nickel. In this instance, the electroplating is performed at 2V in an aqueous Ni bath containing 240 g/l NiSO4.6H2O, 45 g/l NiCl2.6H2O, and 40 g/l H3BO3. In another instance, the mold voids are filled with copper by performing the electroplating in a bath containing 125 g/l CuSO4.5H2O, and 50 g/l H2SO4. The remaining mold precursor is removed by soaking in N-methylpyrrolidone. The copper or nickel that remains provides a solid microneedle in an array.

### Example 2

Different molds are generated using the method of FIG. 2A through FIG. 2C. IP-Dip 780 photoresist served as the mold precursor. Direct laser writing (DLW) using two-photon lithography forms the mold cavities within the mold precursor. The direct laser writing (DLW) is performed at a speed of 50 μm/sec and laser power of 10 mW using the Photonic Professional DLW system (Nanoscribe GmbH, Germany). The resulting mold members are separated from the remaining mold precursor using a developer. A thin layer of Cu is deposited onto the mold members using RF magnetron sputtering to provide electrical conductivity across the surface of the mold. The mold is treated as in Example 1 to electrodeposit copper to plate additional copper onto the surface of the mold cavities to provide hollow microneedles.

### Example 3

The method of FIG. 6A through FIG. 6H is used to generate the mold. IP-Dip 780 photoresist served as the mold precursor. Direct laser writing (DLW) using two-photon lithography forms the mold cavities within the mold precursor. The direct laser writing (DLW) is performed at a speed of 50 μm/sec and laser power of 10 mW using the Photonic Professional DLW system (Nanoscribe GmbH, Germany). The resulting mold members are separated from the remaining mold precursor using a developer. The mold cavities are then conformally coated one monolayer at a time with TiN using an Oxford OpAL Atomic Layer Deposition (ALD) system (Oxfordshire, UK) at 140° C. The deposition is performed by sequentially cycling through the following steps: i) flowing the reactant dose of Titanium Tetrachloride (TiCl4) precursor for 30 ms, ii) purging the system for 5 sec, iii) plasma treatment with an N2/H2 gas mixture (25 sccm/25 sccm) for 10 sec, and iv) purging the system for an additional 5 sec. This process is repeated until a 50 nm thick layer was deposited.

Electrodeposition as described above can be used to provide solid or hollow copper microneedles with a TiN coating.

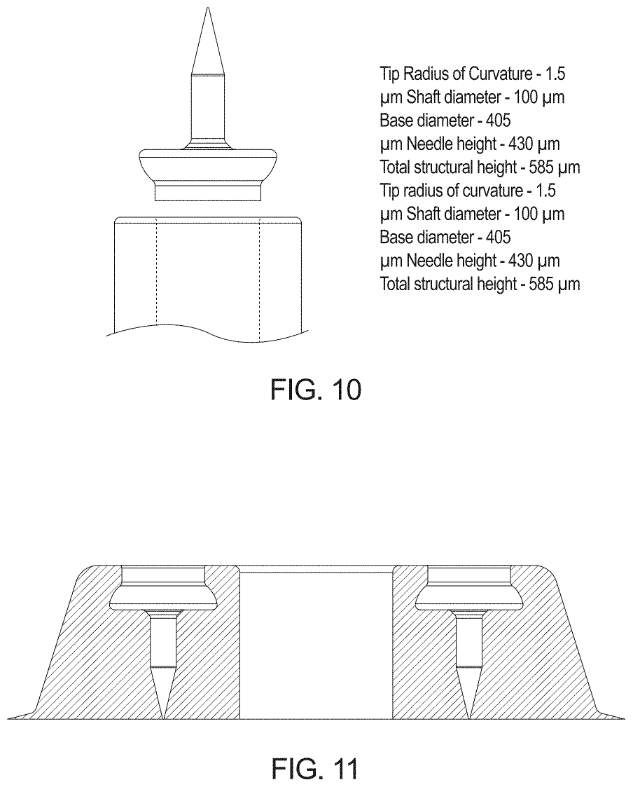

Referring to FIG. 10, the microneedle consists of a shaft of diameter of 100 μm that tapers uniformly with an angle of 13.10- to the tip of 1.5 μm radius. The microneedle's base section diameter is 405 μm. The entire height of the electrochemically deposited needle, including the base part and the needle part is 585 μm, referred to as the total structural height.

To fabricate the microneedles, one of ordinary skill can begin with a (100) single crystal silicon wafer. Thin 100 nm films of titanium and gold are deposited sequentially via electron-beam evaporation (AJA Orion 8E Evaporator System) atop the substrate. Photoresist (IP-S, Nanoscribe GmbH, Karlsruhe, Germany) is deposited atop the gold film via drop-casting. Subsequently, 2PP lithography (3D laser writing is performed using the Photonic Professional GT system (Nanoscribe GmbH, Karlsruhe, Germany) using the Dip-in Laser Lithography (DiLL) configuration with a 25× objective. An example of the cross-section of the mold design can be seen in FIG. 2. After 2PP lithography is complete, the polymeric molds are soaked in a propylene glycol monomethyl ether acetate (PGMEA) solution for 30 min, and are subsequently cleaned in two isopropanol alcohol (IPA) baths of length 20 min each, which completes fabrication of the molds.

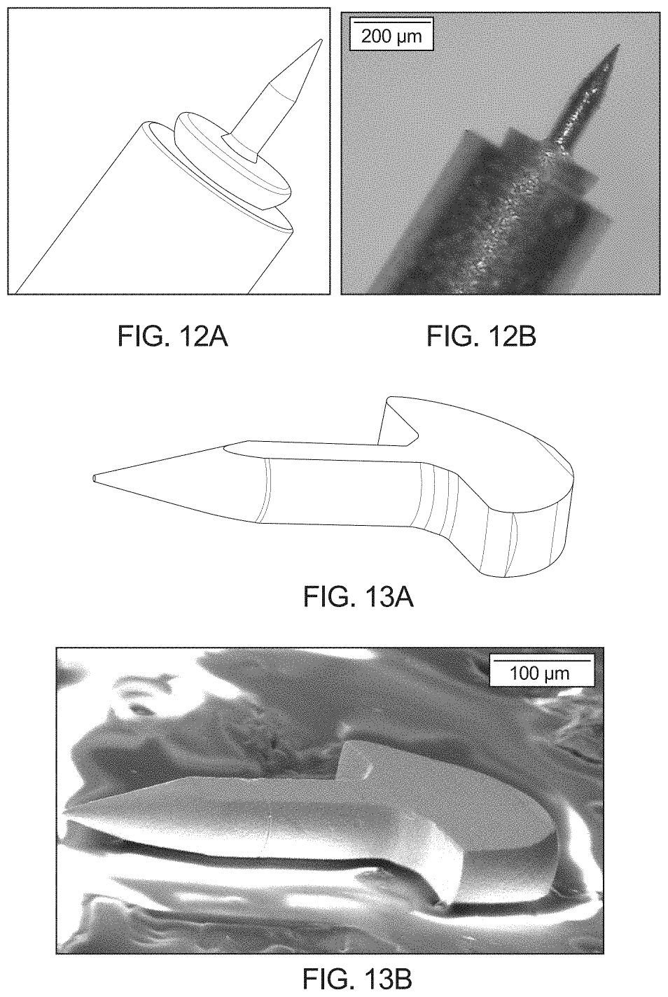

In some embodiments, the fabricated molds are mounted onto a stainless steel rotating disk electrode (RDE) with a polytetrafluoroethylene (PTFE) protector and placed in deionized (DI) water. An electrolyte (Moses Lake Industries, Moses Lake, Wash., USA) is prepared containing 40 g 1-1 copper, 140 g 1-1 CuSO4 and 50 mg 1-1 Cl—. Electrodeposition is performed using a power supply (Metrohm Auto-lab B.V., Utrecht, The Netherlands) that can operate either as a galvanostat or as a potentiostat using a Ag/AgCl reference electrode (Sigma-Aldrich Corporation, MO, USA). Two different methods can be employed in fabricating the needles: (1) Potential controlled (potentiostatic) deposition with a stop function at a limit charge; (2) Current controlled (galvanostatic) deposition with a stop function at a limit potential. After electrodeposition, the copper-filled mold is removed from the RDE and rinsed with DI water and rapidly dried using N2 gas or compressed air. The mold structure is dissolved away to release the needles using Technistrip NF-52 (Technic Inc., Cranston, R.I., USA) that is selective against copper. The microneedles are further cleaned using Reactive Ion Etching (RIE) with 10 sccm CF4 and 50 sccm 02 (PlasmaPro NPG80 RIE, Oxford Instruments, Abingdon, United Kingdom). The copper needles are mounted in a 24-gauge stainless steel blunt hollow hypodermic needle. The microneedle surfaces are then further cleaned via electropolishing using concentrated H3PO4. As shown in FIG. 12 (a) Computer generated rendering of microneedle design is provided, and in FIG. 12 (b) an Optical micrograph of additively manufactured copper needle (with 100 μm shaft diameter) mounted on a 24-gauge stainless steel hollow blunt needle (with nominal 565 μm outside diameter).

The microneedle can be secured to the stainless steel blunt needles with epoxy (Gorilla Epoxy Inc.). The other end of the blunt stainless steel hollow needle can be fitted with a standard Luer Lock fitting to enable connection to medical instruments.

Copper is not a biocompatible material. Hence, after fabricating the microneedles, a nickel thin film (1.5 μm) is blanket coated onto the copper microneedle via electroless deposition (OMG chemicals, MN, USA). A gold thin film (30 nm to 100 nm) (Based on data sheets from Technic Inc.) is then blanket coated atop the nickel film using an immersion deposition method (Technic Inc., Cranston, R.I., USA). In this way, the entire surface of the microneedles is coated with gold, which is a biocompatible material. The Ni interlayer between the Cu and Au prevents the diffusion of Cu into the Au layer. We analyze the Ni and Au coatings using energy-dispersive X-ray spectroscopy (EDS) and backscatter imaging for surface characterization. This analysis is conducted using a scanning electron microscope (Zeiss, Oberkochen, Germany).

To allow ready analysis of the copper microstructure produced by the electrodeposition process, we fabricated microneedles with flat sides in a similar mold structure; an example of such a needle and the design are illustrated in FIG. 4. These microneedles are fabricated with the same process parameters as the round microneedles. The flat microneedles are analyzed using Electron Backscatter Diffraction (EBSD) system (EDAX, NJ, USA). FIG. 13(a) shows a flat needle for microstructural analysis, while FIG. 13(b) shows scanning electron micrograph of the flat-sided needle.

Hartley strain male guinea pigs were used for this study. Immediately following euthanasia, the intact temporal bone of the guinea pig is harvested using blunt dissection. An Osada Electric Handpiece System (Osada, Inc., Los Angeles, Calif., USA) is used to drill and remove the surrounding bone, exposing a clear, wide-angle view of the RWM. The resulting specimen is rinsed with 0.9% saline solution and inspected for gross membrane perforations and fractures of the RWM niche. If perforation of the RWM with the microneedles cannot be performed immediately, the specimen is refrigerated in 0.9% saline solution (up to a maximum of 24 hours) prior to further experimental use. During perforation experiments, small amounts of sterile 0.9% saline solution are applied at regular intervals to keep the membrane from drying.

The microneedles are mounted in a custom-built micromanipulator consisting of: (1) Motorized stage for moving harvested RWM into position (Zaber Technologies Inc., Vancouver, British Columbia, Canada), (2) Motorized linear translator onto which the indenter needle is mounted (Zaber Technologies Inc., Vancouver, British Columbia, Canada), (3) Force transducer with full scale of 98 mN for measurement of axial force exerted on needle during indentation (Transducer Techniques, Temecula, Calif., USA).

A copper microneedle was used to perforate the RWM in-situ while simultaneously measuring the force and displacement (N=4). A separate copper needle is used for each perforation. The perforated RWM is examined via confocal microscopy. The size and orientation of the perforation is recorded and the damage induced into the underlying collagen and elastic fiber matrix is examined.

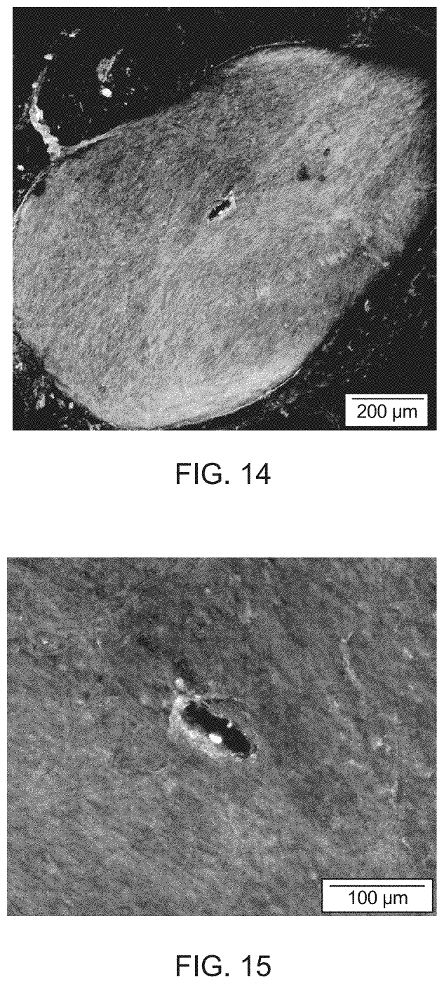

Fully metallic microneedles were fabricated with extreme precision for the goal of perforating the round window membrane. FIG. 12a shows the design of the copper microneedle inserted into the 24-gauge stainless steel hypodermic needle. FIG. 12b shows the fabricated microneedle secured to the hollow stainless steel by a small amount of epoxy glue. FIG. 14 is a SEM micrograph the tip of the needle shown in FIG. 12b.

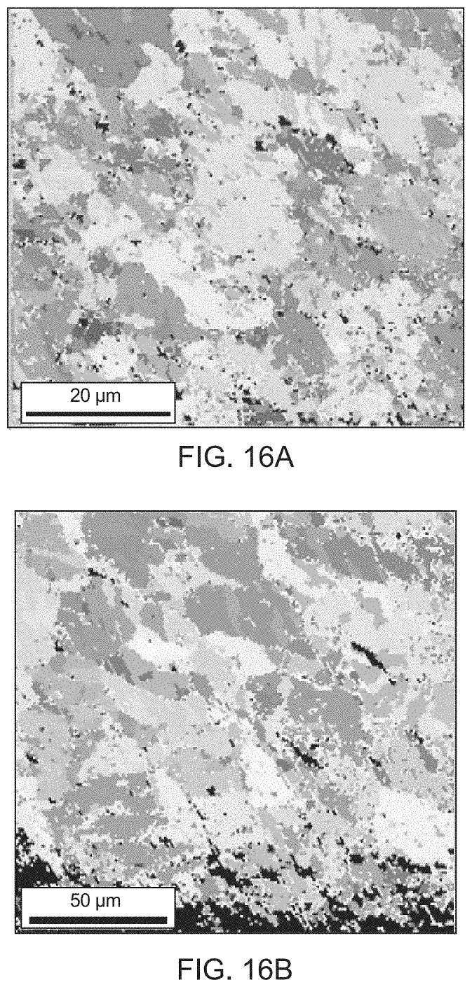

Needles with altered geometries that have flat sides, illustrated in FIG. 13a, are analyzed using EBSD. FIG. 16 shows copper grain microstructure from a region close to the tip of the needle and a region at the base of the needle. The grain sizes average 0.99 μm close to the tip and 1.23 μm close to the base of the needle. This gradient in copper grain size is advantageous because material hardness increases as the grain size decreases via the Hall-Petch effect. Thus a larger grain size is associated with greater ductility, so the copper near the base would be more ductile. A hard microneedle tip is expected to be resilient to damage and a ductile microneedle base is expected to bend rather than break in the event of unanticipated lateral forces during perforation.

Fabricated microneedles are used to perforate guinea pig round window membranes, exerting an average of 3.8 mN of perforation force, with a standard deviation of 0.35 mN. Perforation force was defined as the maximum force that was exerted on the needles during the indentation.

Perforations in the RWM made by microneedles are analyzed using confocal microscopy and are found to have major and minor axes of 75.2 μm and 22.8 μm, respectively, similar to previous results (Aksit et al, 2018). It is also observed from the confocal images that the method of failure of the membrane is fiber-to-fiber decohesion, and not ripping or tearing of the collagen fibers; this is consistent with our earlier study using polymeric microneedles (Aksit et al, 2018). A confocal microscope image of a perforated RWM can be seen in FIG. 15.

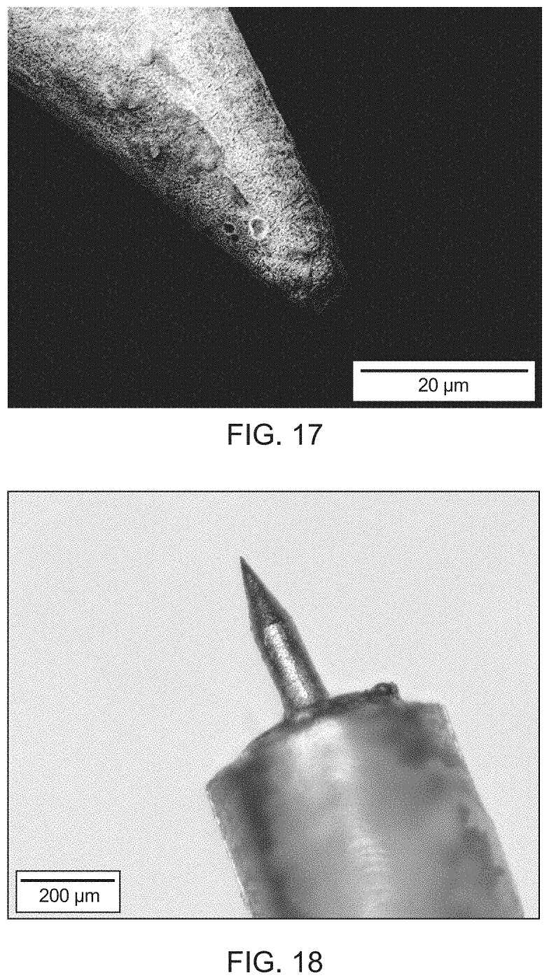

FIG. 17 shows an SEM micrograph of a representative copper microneedle following perforation. There is no discernible damage to the tips of the microneedles, demonstrating the reusability of metallic microneedles post perforation.

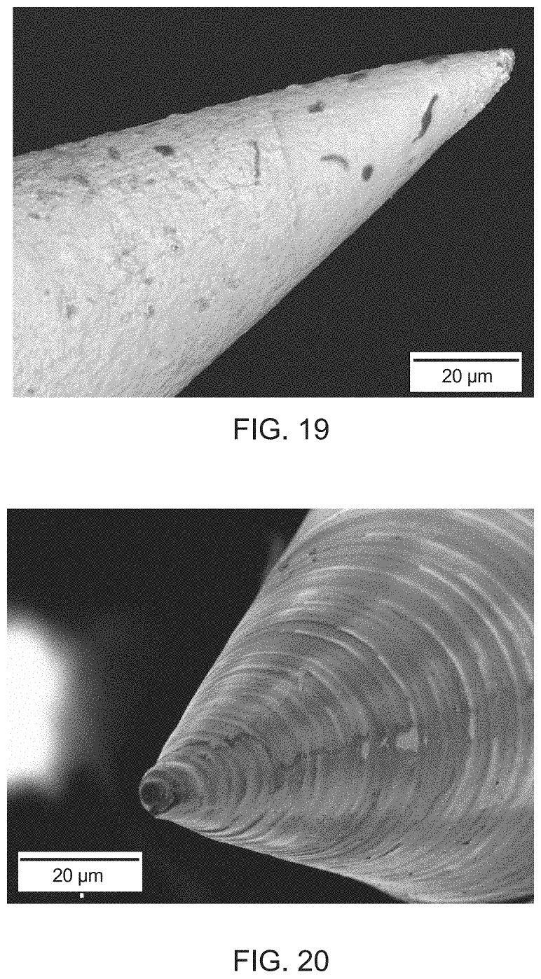

For the copper microneedles subsequently coated with Ni and Au, the EDS and backscatter imaging of the surface show very high and conformal coverage of the surface with Au. A digital microscope image of a gold-coated needle and a backscatter detection image can be seen in FIG. 19. Results of EDS from a needle can be seen in Table 1.

TABLE 1
Results from EDS analysis of the Nickel and Gold coated needles

| | | Mass Norm. | Atomic | Relative error |
|---|---|---|---|---|
| Element | Atomic No. | [%] | [%] | [%] |
| Gold | 79 | 99.06 | 97.07 | 3.76 |
| Copper | 29 | 0.72 | 2.20 | 22.89 |
| Nickel | 28 | 0.22 | 0.73 | 37.46 |

FIG. 20 shows a close-up image of a copper microneedle just after the RIE cleaning step. The tip radius of curvature of the fabricated needles is 1.5 μm. The circumferential lines on the surface of the microneedle taper and shaft are a consequence of the discrete voxel size from 2PP process, leading to individual layers in the mold of the order of micrometers in thickness. These surface features are removed in the electropolishing step of our process. However, these microscale features showcase the detail that can be obtained on the manufactured needles.

The work herein presented discusses the fabrication of fully metallic microneedles that can have complicated geometries and surface designs, along with a demonstration that the microneedles can be used to perforate the guinea pig round window membrane. This methodology provides freedom in the design of needles. However, structures that can be manufactured this way have some geometric limitations. Namely, creation of protruding corners and making rapidly changing cross sectional areas (similar to what was presented in this work) require careful design and control of electrochemical deposition parameters. The realm of the designs that are feasible to produce using this methodology is not dissimilar to regular 3D printing guidelines about overhanging structures (Micallef, 2015). Since the process has extensive design freedom and control over the microscale surface geometry of the microneedles, it is possible to produce a plethora of novel designs, such as hollow microneedles. One other important strength of this technique is its ability to manipulate surface topology, which we know can play an important role in the function of needles. Studies have been made on the effects of blade-like biomimetic protrusions from needles that can decrease insertion force (Izumi et al, 2011; Ma and Wu, 2017).

One other important strength of this technique is its ability to manipulate surface topology, which we know can play an important role in the function of needles. Studies have been made on the effects of blade-like biomimetic protrusions from needles that can decrease insertion force (Izumi et al, 2011; Ma and Wu, 2017). Two different deposition mechanisms were employed to manufacture the microneedles successfully: potential-controlled deposition and current-controlled deposition

The potential-controlled (potentiostatic) method produces full metal needles more reliably and the charge-limit stop ensures stopping at the correct time once the base of the needles are done. However, there is little control over the current density as the deposition proceeds. Using the potentiostatic process means that potentials that could allow for significant hydro gen evolution at the cathode are never reached;

In the current-controlled (galvanostatic) process, a miscalculation of the geometry—especially with complicated geometries with rapid changes in cross sectional area—or misestimation of the current efficiency can mean that the deposition does not occur successfully due to hydrogen evolution that can obstruct the charge flow to the cathode, and increase the current density. Hence, the galvanostatic process is always used in conjunction with a potential limit, which ensures that low potentials that could evolve significant amounts of hydrogen are not reached. The advantage of using the galvanostatic method is the fact that the user has complete control over the current density at every point throughout the deposition.

Using either of these methods, it is possible to change parameters of deposition, such as additive concentrations, temperature, and current density to induce an effect on the microstructure, thereby having an effect on the microneedle's mechanical properties (Kelly et al, 1999). These effects can be experimentally verified via nanoindentation and can be visualized using EBSD. The goal would be to manipulate the microstructure of microneedles such that while the tip portion is hard and can hold an edge after many perforations, the base portion is softer and would bend rather than break. Electroless deposition of Ni and subsequent immersion coating with Au is used to make the microneedles biocompatible. Although it is shown, through EDS and backscatter imaging, that the microneedles are coated with good conformality, the strength and survivability of these coatings.

Fully metallic single microneedles were manufactured by electrochemical deposition of copper into molds made via two-photon polymerization lithography. The needles were ultra-sharp, their tip of radius of curvature was found to be 1.5 μm. The needles were mounted on standard blunt 24 gauge stainless steel syringe needles and coated with layers of electroless nickel and immersion gold for biocompatibility. Success of the deposit was verified via EDS. Copper microneedles successfully perforated the guinea pig round window membranes, resulting in a mean perforation force of 3.8 mN

Confocal microscopy of the membranes suggested that the method of failure of the membrane was fiber-to-fiber decohesion. Other than the spatial resolution, 2PP lithography enables manufacturing structures with great design freedom. As an example, we demonstrate the use of electrochemical deposition into tem plates/molds that have cross-sectional areas which increase by four orders of magnitude from the tip of the needle to its base.

In another embodiments, microneedles are fabricated by applying pyrolysis to the 3D printed polymeric microneedles. Pyrolysis is a technique applied to convert carbon-rich polymers to functional high-carbon solids at elevated temperatures (e.g., 400° C. to 1800° C.) under an inert atmosphere. Without being bound to a particular theory, organic molecules in a carbon-rich polymer break down during pyrolysis, and the resulting gases and volatile products leave the sample and non-volatile residues form large disordered molecules that typically become richer in carbon (Singh et al, 2002). The resulting glassy carbon material is sp² bonded with properties including high temperature resistance, high hardness, low density, low electrical resistance, low friction, and low thermal resistance. These physical, mechanical, and chemical properties are strongly influenced by the heat treatment process (Schueller et al, 1997), which presents the opportunity to tailor the properties of the pyrolyzed material. Under carefully selected techniques, pyrolyzed materials can be biocompatible (Sharma, 2018; Schueller et al, 1997) and as such have been widely used in medical devices (Bokros, 1983; Salkeld et al, 2016).

During the pyrolysis, a carbon-rich polymer shrinks significantly while generally retaining its overall shape, which creates the opportunity to fabricate very small structures. However, pyrolysis occurs most rapidly on the surface of the polymer which can lead to the development of tensile stresses at the surface that can induce fracture. In addition, non-spatially-uniform pyrolysis especially of complex shapes can lead to non-uniform shrinkage that can cause significant changes in shape.

Exemplary precursor materials include SU-8 and IP-S photoresist. For example, SU-8 photoresist is a well-known precursor material for pyrolysis and recipes have been developed for pyrolyzing it (Wang et al, 2005; Hassan et al, 2017; Zhang et al, 2011). Among those recipes, there exists one for fabricating glassy carbon microneedles derived from pyrolysis of SU-8 (Mishra et al, 2018). Pyrolysis of SU-8 is based on the SU-8 patterning technique. As a result, the design freedom of this method is at the same level as the UV-cured SU-8 microneedles. While IP-S is also a carbon-rich photoresist like SU-8, relatively few pyrolysis studies have been reported. One study used IP-S pyrolysis to fabricate carbon electrodes (Yang et al, 2018), showing feasibility. However the geometries explored in that study are cone and sphere structures, which are relatively simple and less bulky shapes compared microneedles that can be mounted in a surgical tool. Thus, isotropic pyrolysis of complex 3D printed IP-S precursors remains challenging.

In accordance with the disclosed subject matter, a solution to this challenge is disclosed herein, in which a parametric study was conducted to fabricate pyrolyzed microneedles. The outcome microneedles have high design freedom and good biocompatibility, expand the material category of 2PP-based microneedles, and can be fabricated in a reproducible and scalable manner. The carbon microneedles fabricated according to the techniques described herein have demonstrated utility by perforating the RWM of guinea pigs (GP) to confirm that they are sharp and robust enough to make microperforations across RWM.

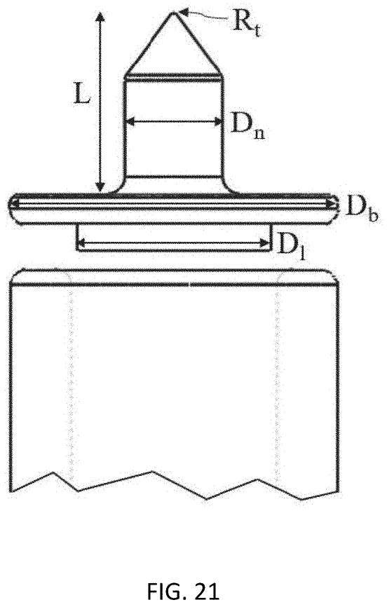

A solid microneedle 200 is shown in FIG. 21. Microneedle 200 includes a tip having a radius Rₜ and tapers at an angle a to a constant shaft diameter Dₙ, with a taper-plus-shaft length of L and a base with maximum diameter D_{b} designed to fit into the lumen with diameter, Dₗ, of a blunt stainless-steel needle. A Luer lock is affixed to the other end of the stainless-steel needle (not shown).

Polymeric microneedles with Rₜ=500 nm, Dₙ=100 μm, α=18°, L=200 μm, D=320 μm and Dₗ=170 μm performed in vitro studies of GP RWM perforation. The characteristics of the microneedle design were modified from the GP microneedles to perforate in vitro the stronger and thicker human RWM, with Rₜ=5 μm, α=60°, Dₙ=150 μm, L=350 μm, D_{b}=300 μm and Dₗ=160 μm.

Hollow microneedles were 2PP printed with Rₜ=500 nm, Dₙ=100 μm, α=24°, L=435 μm, and 35 μm diameter lumen and mounted atop a 30 gauge blunt hollow stainless-steel needle with D_{b}=310 μm and Dl=139 μm. 1 μL of GP perilymph across the RWM was aspirated in vivo for proteomic analysis.

A fully-metallic microneedle has Rₜ=1.5 μm, Dₙ=100 μm, α=13.1°, L=430 μm, Db=405 μm and Dₗ=310 μm and was tested in vitro in a GP RWM.

The microneedles from pyrolysis followed the same general design. The dimensions of the 2PP printed microneedles can be prescribed, with Rₜ=7.5 μm, Dₙ=100 μm to 120 μm, α=10° to 24°, L=600 μm to 850 μm, D_{b}=600 μm and Dₗ=280 μm. The dimensions of the pyrolyzed microneedle depend on the pyrolization parameters. It was mounted on a Gauge 32 syringe tip.

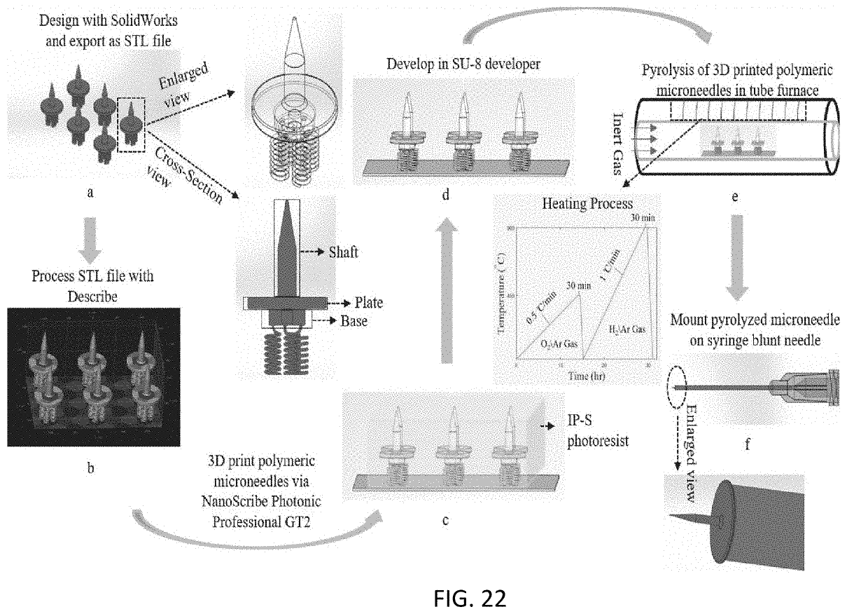

The 2PP printed microneedles were written in Photonic Professional GT system (Nanoscribe GmbH, Germany). The equipment has finest XY resolution of 400 nm, vertical resolution of 1000 nm, maximum X and Y dimensions of 100 mm, and maximum height of 8 mm (Nanoscribe GmbH, 2018). Based on the general design of the microneedles, the design included modifications to survive the pyrolysis process. Hole cut was inserted in the polymeric microneedle base to increase its surface area-to-volume ratio. A large surface area-to-volume ratio ensured that gas generated during pyrolysis process can escape across the surface. Spring structures were inserted under the base to decouple the structure from substrate so that isotropic shrinkage could be achieved. To avoid structural detachment from the substrate during shrinkage, a flat surface at bottom of the springs and chamfers with radius of 10 μm and 30° were designed. Fillets were used at the edges, connections between shafts, bases and tips to reduce stresses. The design was done with computer-aided design software SolidWorks (Dassault Systems SolidWorks Corporation, USA) as shown in FIG. 22 and exported as Stereolithography (STL) files.

FIG. 22 illustrates the stages in the fabrication of pyrolyzed microneedles. At step (a), design and export as STL file; at step (b) process file; at step (c) 3D print of the polymeric microneedles; at step (d) cleaning uncured photoresist; at step (e) pyrolyze 3D printed microneedles; and step (f) mounting the pyrolyzed microneedles.

The STL file was first imported into software DeScribe provided by Nanoscribe (Nanoscribe GmbH, 2018) to prepare the CAD model for printing. Printing parameters were selected as fixed slicing distance of 0.5 μm, solid fill mode, hexagonal splitting mode and lexical block order. Single crystal Silicone (100) wafer served as substrate. It was rinsed with acetone and isopropyl alcohol (IPA). The IP-S photoresist was drop cast on the substrate and polymeric microneedles were 2PP 3D laser written by the Photonic Professional GT system. The polymerized structures were then developed in 1-Methoxy-2-propanol acetate for 20 min and subsequently immersed in IPA bath for 5 min.

The microneedles were pyrolyzed in Thermo Scientific Lindberg/Blue M TF55030A tube furnace (Hogentogler, USA). The heating procedure is illustrated at step (e) of FIG. 22. It followed flowing Oxygen (5%)/Argon gas (101 kPa), ramping up temperature from room temperature to 450° C. at 0.5° C. min⁻¹, holding at 450° C. for 30 min, ramping down to room temperature at 10° C. min⁻¹, changing flowing gas to Hydrogen (5%)/Argon gas (101 kPa), ramping up to 900° C. at 1° C. min⁻¹, holding at 900° C. for 30 min, and finally ramping down to room temperature at 10° C. min-.

The carbonized microneedle was caught up with tweezers' tip, manually inserted onto a commercially available Gauge 32 syringe tip (Sterile standard blunt needle) and adhered with epoxy as shown in step (f) of FIG. 22 (syringe blunt needle CAD model from GrabCAD (Baycura, 2018)). Honey was used on the syringe tip and tweezers' tip to ease the manipulation resulting from microneedle's tiny size. The commercially available syringe tip was chosen as the mounting base because of its familiarity to clinicians. Finally, honey and other residues on the microneedle were cleaned with IPA.

A Scanning Electron Microscope (SEM, Zeiss, Germany) was used to characterize the microneedles' geometry. Material composition characterization of the microneedles before and after pyrolysis was done by Energy-dispersive X-ray spectroscopy (EDS, Bruker, UK). Raman spectroscopy analysis (Renishaw, UK) was utilized to determine crystallinity. Mechanical properties of pyrolyzed carbon were measured with Nanoindenter G200 (KLA, USA). To characterize the pyrolyzed material's viscoelasticity, creep test was executed by prescribing 100 mN constant force on a 22 μm diameter micropillar with a 1 mm diameter flat punch for 10000 s. To measure Young's modulus, each of six 22 μm diameter micropillars was compressed to 150 mN at 0.01 s⁻¹ strain rate. The force was held for 10 s and unloaded at 5 mN s⁻¹. For compressive strength, the indenter tip was set to load each of 11 micropillars until 25 μm depth at 0.001 s⁻¹ strain rate. The 22 μm diameter micropillars were chosen for creep and compression tests because this diameter matched the microneedles' shaft diameter. The diameter was measured at half height of the micropillar.

In vitro perforation experiments were done with mounted microneedles. The protocol used herein was substantially the same as the perforation tests performed in Aksit, A., Arteaga, D. N., Arriaga, M. et al., “In-vitro perforation of the round window membrane via direct 3-D printed microneedles.” Biomed Microdevices 20, 47 (2018) (hereinafter “Aksit et al., 2018”) with 2PP fabricated microneedles. Hartley strain male guinea pigs were euthanized and their intact temporal bones were harvested with blunt dissection immediately afterwards. Osada Electric Handpiece System (Osada, Inc., USA) was utilized to drill and remove the surrounding bone around RWM in order to get a clear view. Then the sample was rinsed with 0.9% saline solution and kept refrigerated in the solution (up to a maximum of 24 hours) until the perforation test. During the experiment, drops of sterile 0.9% saline solution were applied at regular intervals to keep the membrane from drying. We used the microindenter (Aksit et al, 2018) to make the perforations. The microindenter has a motorized stage for manipulating harvested RWM's position, motorized linear translator to control the needle and force transducer for measurement of axial force exerted on the needle during indentation. The force transducer can measure up to 98 mN. The holes perforated were scanned with confocal laser scanning microscope (Zeiss, Germany) and the microneedles after perforation were imaged with SEM to determine damage.

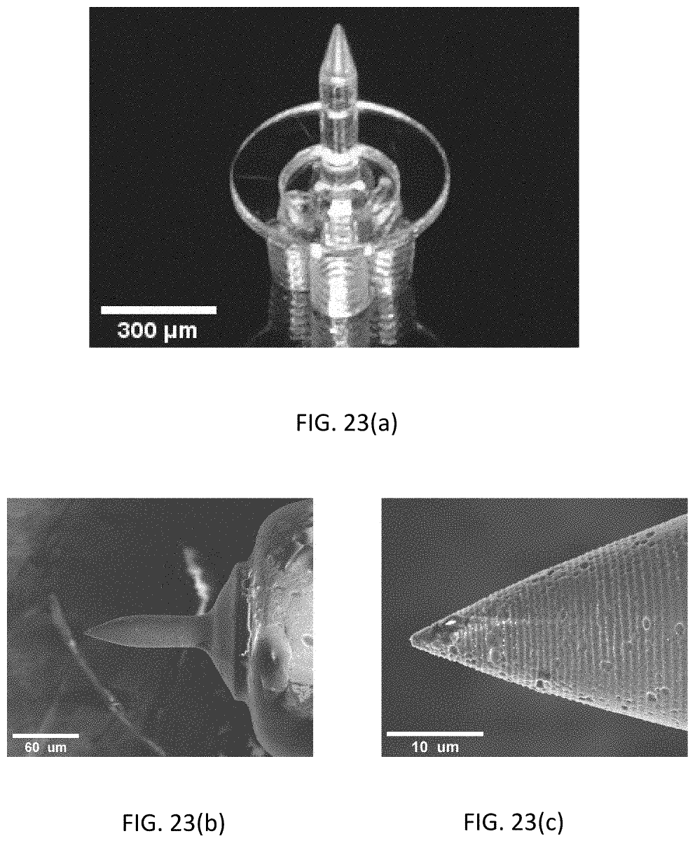

The recipe developed enables consistent fabrication of pyrolyzed microneedles. FIGS. 23(a)-(c) demonstrate an example of a 3D printed polymeric microneedle, its corresponding pyrolyzed feature mounted on the syringe tip and enlarged tip view. FIG. 23(a) is an optical image of a 3D printed polymer microneedle. FIG. 23(b) illustrates a SEM image of the corresponding pyrolyzed microneedle mounting on Gauge 32 stainless steel syringe tip. FIG. 23(c) is a SEM image of the enlarged view on the pyrolyzed microneedle's tip. The polymeric microneedles' shafts have diameters of 100 μm, length of 600 μm and tip radius of 7.5 μm. Their diameters decrease to 23±2 μm (n=6), length to 120±10 μm and tip radius to 1 μm after pyrolysis, achieving shrinkage factors of 0.77±0.02 and 0.80±0.02 respectively for diameters and length. Microneedles with other dimensions are also 3D printed and pyrolyzed to present the recipe's compatibility with various dimensions. The microneedles with larger diameters tend to shrink less than the smaller ones. The dimensions before and after pyrolysis are reported in Table 2.

TABLE 2
Dimensions of microneedles before and after pyrolysis

| IP-S Microneedles | | Glassy Carbon Microneedles | | Shrinkage Factor | |
|---|---|---|---|---|---|
| Diameter (μm) | Length (μm) | Diameter (μm) | Length (μm) | Diameter | Length |
| 120 | 600 | 32 | 120 | 0.73 | 0.80 |
| 150 | 600 | 48 | 140 | 0.68 | 0.77 |
| 100 | 850 | 25 | 195 | 0.75 | 0.77 |

Shrinkage factor (SF) is calculated as:

$$SF = \frac{{D}_{p} - {D}_{c}}{{D}_{p}} \qquad ( 1 )$$

where Dₚ is the dimension of polymeric microneedle and Dc is the dimension of carbon microneedle. The outer diameter of the pyrolized needle was found to be between about 20-50 μm, the length of the pyrolized needle was found to be between about 100 to about 200 μm, and the tip having a radius of about 1 μm.

The EDS results are given in Table 3. Nitrogen was not found in the pyrolyzed microneedle, indicating the nitrogen component has been removed completely during the heating process. Oxygen atomic percentage decreases significantly from 19.69% to 3.85% showing that only a small amount of oxygen is left. The microneedle is mainly composed of carbon with an atomic percentage of greater than 85% after pyrolysis. In some embodiments, the microneedle is mainly composed of carbon with an atomic percentage of greater than 90% after pyrolysis. In some embodiments, the microneedle is mainly composed of carbon with an atomic percentage of greater than 94% after pyrolysis. In some embodiments, the microneedle includes oxygen with an atomic percentage of less than 5% after pyrolysis. In some embodiments, the microneedle includes silicon with an atomic percentage of less than 5% after pyrolysis. As shown in Table 3, the atomic percentage of carbon is 94.22%. There is also silicon component detected, and without being bound to a particular theory, it is presumed to result from the silicon substrate.

TABLE 3
EDS analysis results for IP-S and pyrolyzed carbon microneedles

| Atomic (%) | IP-S microneedle | Glassy Carbon Microneedle |
|---|---|---|
| Carbon | 70.70 | 94.22 |
| Oxygen | 19.69 | 3.85 |
| Nitrogen | 1.64 | |
| Silicon | 0.06 | 1.77 |

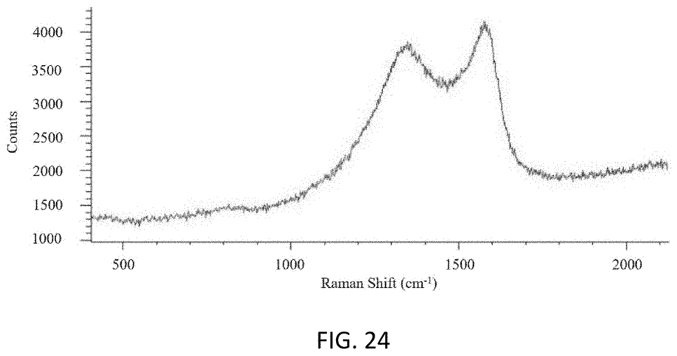

Raman spectroscopy analysis in FIG. 24 shows two peaks at about 1350 cm⁻¹ and 1580 cm⁻¹. The first peak is around the literature D-band value (1357 cm⁻¹) and the second peak matches the G-band value (1580 cm⁻¹). The G-band is related to sp²-hybridized carbon atoms and well-crystallized graphite with a small particle size shows a D-band in addition to the G-band (Knight and White, 1989). The intensity ratio of D-band over G-band (ID/IG) is around 0.93, which is within glassy carbon's intensity ratio (0.91 to 1.32) reported in literature (Knight and White, 1989; Mishra et al, 2018). Overall, the EDS and Raman spectroscopy results verify that the resulting material is glassy carbon.

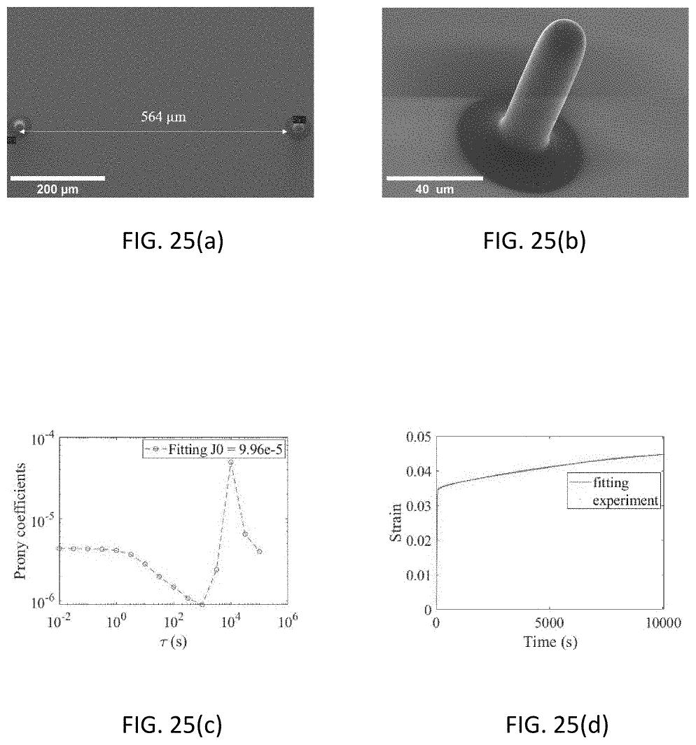

The glassy carbon derived is viscoelastic, as illustrated in the viscoelastic characterizations of FIGS. 25(a)-(d). Creep tests are performed on pyrolyzed micropillars spacing more than 550 μm on a silicon substrate as shown in FIG. 25(a). In an exemplary analysis technique, and assuming the micropillar is load free prior to time t=0, at which a ramping up stress σ(t) is applied until t=t₁, the strain ε(t) can be expressed as:

$$ɛ ( t ) = {\int }_{0}^{t} J ( t - ξ ) \frac{d σ ( ξ )}{d ξ} d ξ \qquad ( 2 )$$

where

$$\frac{d σ ( ξ )}{d ξ}$$

is the stress rate and J(t) is the compliance function of the material defined as:

$$J ( t ) = {J}_{0} + {J}_{0} {\sum }_{i = 1}^{n} {p}_{i} ( 1 - {e}^{- i \text{/} π} ) \qquad ( 3 )$$

where J₀ is the instantaneous creep compliance of the material, pi is the i^{th} Prony constant and r is the i^{th} Prony retardation time constant. For t₁<t≤t₂, at which a constant stress is prescribed, the hereditary integral can be modified as:

$$ɛ ( t ) = {\int }_{0}^{{t}_{1}} J ( t - ξ ) \frac{d σ ( ξ )}{d ξ} d ξ + {\int }_{{t}_{1}}^{t} J ( t - ξ ) \qquad ( 4 )$$

With above equations, sign control method (Bradshaw and Brinson, 1997) is implemented in Matlab (Mat-works, USA). The retardation times are exemplary and chosen for mathematical convenience.

FIG. 25(a) is a SEM image of a representative pyrolyzed micropillars array on a silicon substrate, FIG. 25(b) is a SEM image of the enlarged tilted view of a micropillar (diameter=22 μm, height=70 μm). Prony representations are numerically approached by fitting the strain vs. time response with hereditary integral method (Chen, 2000), generalized Kelvin model and discrete spectrum method (Brinson and Brinson, 2008). Engineering strains and stresses are used for all experiments. Detailed fitting process is discussed in the support material. FIG. 25(c) shows the prony series representation of the material. The viscoelastic response is well captured with this method by comparing the fitting and experiment results (FIG. 25(d)).

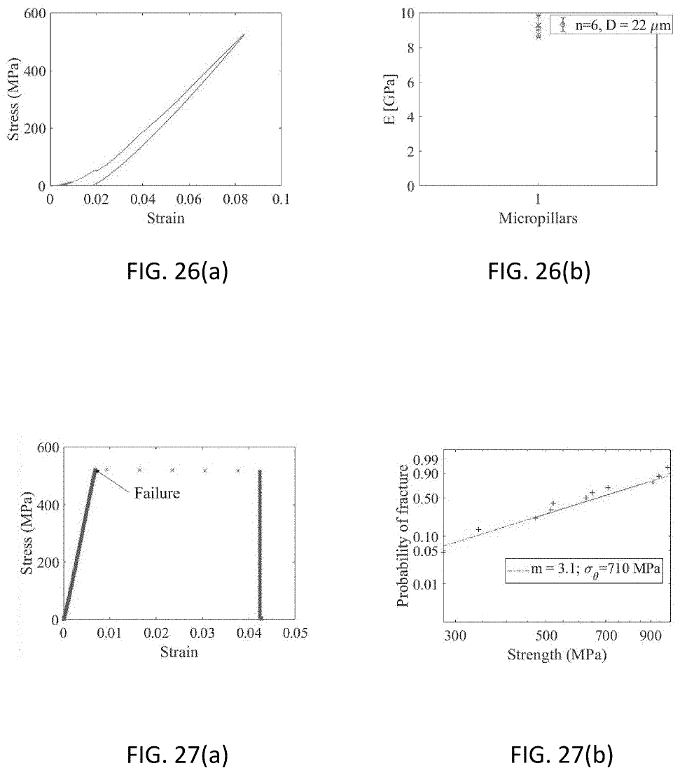

A representative stress-strain curve for measuring Young's modulus is shown in FIG. 26(a). The Young's modulus is found to be about 9 GPa by calculating the slope of first 5% to 25% of the unloading curve. Measured values are 9.1 GPa with 95% confidence interval (CI) from 8.6 GPa to 9.6 GPa (n=6) as plotted in FIG. 26(b). The tested micropillars suddenly shatter into pieces when compressing to failure as shown in FIG. 27(a), demonstrating catastrophic brittle fracture. The compressive strength values of brittle material depend on flaws containing in the micropillars. Thus, a two-parameter Weibull distribution model with maximum likelihood estimator, discussed in supporting material, is applied to fit the compressive strength data as shown in FIG. 27(b). The measured highest load the micropillar can withstand is 358 mN and the corresponding compressive strength is 990 MPa. The Weibull modulus is 3.1 with 95% CI from 1.9 to 4.9 and characteristic strength a6 is 710 MPa with 95% CI from 580 MPa to 870 MPa.

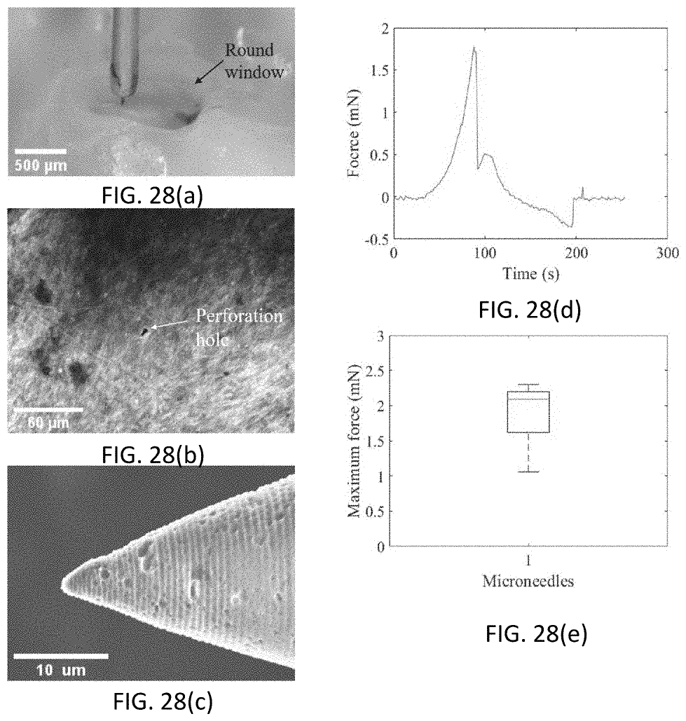

In order to assess the performance of the glassy carbon microneedles perforating the RWM, a total number of 6 microperforation experiments were performed with the pyrolyzed microneedles. One example experimental result of the perforations is demonstrated in FIGS. 28(a)-(e). As shown in FIG. 28(b), the perforations are oval shaped. FIG. 28(d) illustrates that the force exerted on the microneedles first increases and then starts to decrease when the membranes are completely penetrated. The maximum forces experienced are in range of 1.1 mN to 2.3 mN as shown in the box plot of FIG. 28(e). The perforation results correspond to those perforated with polymeric microneedles, Aksit et al (2018) 3D printed before. Pyrolyzed microneedles are able to penetrate the RWM successfully without ripping or tearing the RWM. Neither, the pyrolyzed microneedles are not damaged as illustrated in FIG. 28(c). The load this glassy carbon material can hold is hundred times of the resistive force resulting from the membrane.

After several heating process trials, holding temperatures (450° C. & 900° C.) are kept the same as in the recipe developed by Zhang et al (2011) but a slow temperature ramp rate (0.5° C. min⁻¹) during pyrolysis is found crucial to prevent the bulky polymeric structure from explosion. In this case, non-carbon atoms are broken down into compounds with lower molecular rate at a slow rate, therefore, have sufficient time to diffuse across the structure. Another important factor to sustain the structure during pyrolysis is surface area to volume ratio (SVR) of the structure. The SVR is calculated individually for base, plate and shaft of the microneedle as indicated in step (a) of FIG. 22. The surface area calculated only includes surfaces that are exposed to the environment. In order to have large enough surface area for degassing, the SVR needs to be greater than 0.025 for this pyrolysis recipe. However, SVR of a solid cylindrical base would be smaller than 0.025 if it has the proper dimension to fit into the Gauge 32 needle after shrinkage. Hence, the base is made hollow to increase the SVR which can be controlled by varying the hole diameter. While maintaining the high SVR for degassing, the thickness of the base and plate should not be too thin to sustain forces taken during shrinkage. Thus, the SVR should be less than 0.04.

In order to successfully perforate RWM, the microneedle's tip radius of curvature must be much less than the RWM's thickness which is ranging between 10 μm to 30 μm (Aksit et al, 2018). The 3D printed polymeric microneedle's tip may be distorted during pyrolysis if its radius of curvature is directly specified as a small value that is adequately sharp such as 1 μm. After iterations, 7.5 μm is finally assigned for the polymeric microneedle's tip radius so that it can uniformly shrink with the rest to a radius of 1 μm.

Higher compressive strength (1.2 GPa to 7 GPa) and Young's modulus ranges (15 GPa to 47 GPa) have been reported for literature values of glassy carbon (Albiez and Schwaiger, 2019; Bauer et al, 2016). One possible reason for this is that the micropillars tested in this work are in larger dimensions compared to the ones used in literature, in which more defects may result from the pyrolysis process. By switching to vacuum furnace instead of flowing inert gas during pyrolysis and further exploring the 3D printing parameters including contour counts and slicing distances, glassy carbon material with a lower defect concentration can possibly be achieved. The ability to fabricate glassy carbon with good biocompatible, electrical conductivity and custom shapes suggests potential applications in medical implants, heart valves, electrodes and so on.

The fabrication method presented herein introduces a consistent way to convert 3D printed polymeric microneedles into ultra-sharp glassy carbon microneedles with tip radius of 1 μm and good biocompatibility. The pyrolyzed microneedles shrink by a factor up to 80% of the polymeric ones. EDS and Raman analysis results confirm that the remaining material after pyrolysis is glassy carbon. It contains 94.22% carbon and shows D-band and G-band at 1350 cm-1 and 1580 cm⁻¹ respectively. Viscoelasticity is found in the pyrolyzed glassy carbon material. A discrete spectrum of retardation time for the material is derived by fitting the creep test result with generalized Kelvin model. Results of compression tests on 22 μm micropillars show that this pyrolytic carbon has Young's modulus of 9.1 GPa with 95% CI from 8.6 GPa to 9.6 GPa (n=6), and fails in catastrophic brittle way. Its compressive strength is in range 280 MPa to 990 MPa, depending on flaw level of the samples. Measured compressive strength values are fitted with Weibull distribution.

The two-parameter Weibull distribution (ASTM, 2003) is written as:

$${P}_{j} = 1 - exp [ - {( \frac{σ}{{σ}_{0}} )}^{m} ] \qquad ( 5 )$$

where P_{f} is probability of failure, σ is measured uniaxial compressive strength of the glassy carbon, m is Weibull modulus, and σ_{θ} is Weibull characteristic strength. The two parameters are approached with maximum likelihood estimates, using Matlab command wblfit (The MathWorks Inc., 2020) to solve the simultaneous equations:

$${\hat{σ}}_{0} = {[ \frac{1}{n} {\sum }_{i = 1}^{n} {σ}_{i}^{m} ]}^{\frac{1}{n}} \\ \hat{m} = \frac{n}{\frac{1}{{\hat{σ}}_{0}} {\sum }_{i = 1}^{n} {σ}_{i}^{m} log {σ}_{i} - {\sum }_{i = 1}^{n} log {σ}_{i}} \qquad ( 6 )$$

Where \hat{ }σ_{θ} and m\hat{ } are unbiased estimators of the parameters σ_{θ} and m. Since m and σ_{θ} are positive parameters, the log transformation is used to obtain the 95% confidence interval (Meeker and Escobar, 2014).

The Weibull modulus is about 3, e.g., 3.1 with 95% CI from 1.9 to 4.9 and characteristic strength a6 is 710 MPa with 95% CI from 580 MPa to 870 MPa. The pyrolyzed microneedles are manually mounted on Gauge 32 syringe tip and utilized to perforate harvested GP's RWM. Perforation tests illustrate that the glassy carbon microneedles are sharp and robust enough to introduce oval shaped holes in RWM. After perforation experiments, confocal images of the RWM show no tearing or ripping and the SEM images of the microneedles indicate no damage on the tips or shafts. The resistive force exerted by the membrane on the needle is around 2 mN, which is two orders of magnitude smaller compared to the force that the glassy carbon column can take.

While the above description has focused on using the microneedle claimed and embodied herein for the round window membrane application, one of ordinary skill in the art would appreciate that the pyrolyzed microneedle has widespread use for other applications, such as but not limited to ophthalmological or transdermal drug delivery.

What is claimed is:

1. A microneedle for perforation of an anatomic membrane, the metal microneedle having a fully metal body, the body comprising a proximal portion, a distal portion and a length there between, the proximal portion including a longitudinal shaft having an outer diameter of about 100 micron and a microneedle height of about 450 micron, wherein microneedle is configured to create a perforation of an anatomic membrane that self-closes within a period of time, wherein the period of time is one week or less.

2. The microneedle of claim 1 having a maximum tip diameter of about 10-20 microns.

3. The microneedle of claim 1 wherein the microneedle is solid.

4. The microneedle of claim 1 wherein the microneedle is hollow.

5. The microneedle of claim 1 configured with a taper along its length.

6. The microneedle of claim 5 wherein the taper comprises a gradual taper having a gradual decrease in diameter along the length of the microneedle.

7. The microneedle of claim 5 wherein the taper comprises a stepped taper with abrupt changes in diameter that serve as reinforcing ribs or ledges.

8. The microneedle of claim 1 wherein the base of the distal portion may comprise one or more projections or barbs that engage the distal side of the membrane after penetration through the membrane and is held in place thereby.

9. An array comprising a plurality of the microneedle of claim 1.

10. A medical device comprising a plurality of microneedles of claim 1 coupled to a base that is configured to physically engage a driver device capable of creating temporary perforations in an anatomic membrane.

11. The medical device of claim 10 wherein the membrane is the round window membrane of an inner ear.

12. The medical device of claim 10 wherein the medical device and the driver comprise separate components that are engaged to each other to define a modular system.

13. A system for delivering therapeutic agent to the inner ear of a subject comprising an instrument for accessing the round window membrane; a plurality of microneedles of claim 1, with sufficient rigidity to perforate the round window membrane; and a driver, wherein the plurality of microneedles is coupled to the driver.

14. The system of claim 13 further comprising an indicator disposed along the system to indicate when the membrane is fully penetrated by the microneedles.

15. The system of claim 13 further comprising an aspirating lumen within at least one microneedle which is connected to a suction device.

16. A method of delivering a therapeutic agent through an anatomic membrane, the method comprising positioning at least one microneedle of claim 1 proximate the membrane wherein the microneedle is configured to penetrate the membrane; perforating the membrane; and dispensing a therapeutic agent at said perforation(s).

17. The method of claim 16 for delivering a therapeutic agent into the cochlea comprising positioning at least one microneedle as described herein proximate the round window membrane wherein the microneedle is configured to penetrate the round window membrane; perforating the round window membrane; and dispensing a therapeutic agent at said perforation(s).

18. A method for manufacturing a metal microneedles of claim 1, comprising:
preparing a mold comprising at least one mold cavity on a conductive substrate by multiphoton lithography of a photoresist material;
electrodepositing metal in the mold cavity to provide a microneedle; and
removing the mold and conductive substrate from the microneedle.

19. The method of claim 18 wherein the microneedle is solid.

20. The method of claim 18 wherein the microneedle is hollow.

21. The method of claim 18 wherein the mold comprises a plurality of mold cavities and an array comprising a plurality of microneedles is provided.

22. The method of claim 18 further comprising depositing a layer of conductive material on the surface of the mold cavity by physical vapor deposition (PVD) prior to electrodeposition.

23. The method of claim 18 wherein PVD comprises sputter deposition, cathodic arc deposition, electron beam heating, chemical vapor deposition or atomic layer deposition.

24. The method of claim 18 wherein the microneedle is further treated after mold removal to provide a finished product by polishing, etching and/or coating.

25. The method of claim 24 wherein the exterior of the microneedle is coated with a thin layer of TiN by atomic layer deposition.

26. The method of claim 24 wherein the exterior of the needle is coated with a therapeutic agent, optionally encapsulated by a biodegradable polymer.

27. A pyrolyzed microneedle comprising
a pyrolyzed body having a tip, base, and length therebetween, wherein the body comprises an atomic percentage of carbon of greater than 85%.

28. The pyrolyzed microneedle of claim 27, wherein the atomic percentage of carbon is greater than 90%

29. The pyrolyzed microneedle of claim 27, wherein the atomic percentage of carbon is greater than 94%

30. The pyrolyzed microneedle of claim 27, wherein the pyrolized body is free of nitrogen.

31. A pyrolyzed microneedle comprising
a pyrolyzed body having a tip, base, and length therebetween, wherein the body comprises an atomic percentage of about 94% and an atomic percentage of oxygen of less than 5% percent.

32. The pyrolyzed microneedle of claim 31, wherein the outer diameter of the pyrolyzed body is between about 20 to 50 μm.

33. The pyrolyzed microneedle of claim 31, wherein the length of the pyrolyzed body is between about 100 to about 200 μm.

34. The pyrolyzed microneedle of claim 31, wherein the pyrolyzed body has a tip of radius of about 1 μm.

35. The pyrolyzed microneedle of claim 31, wherein the microneedle formed from carbon, oxygen and silicon atoms, and is substantially free of other atomic elements.

36. A pyrolyzed microneedle comprising
a pyrolyzed body having a tip, base, and length therebetween, wherein the pyrolyzed body comprises an atomic percentage of carbon of greater than 85%, and the outer diameter of the pyrolized microneedle is about 20 micron and the length is less than 200 micron.

37. The pyrolyzed microneedle of claim 36, wherein the pyrolyzed body further comprises oxygen and silicon in an atomic percentage.

38. The pyrolyzed microneedle of claim 37, wherein the atomic percentage of the oxygen is less than 5%.

39. The pyrolyzed microneedle of claim 37, wherein the atomic percentage of the silicon is less than 5%.

40. The pyrolyzed microneedle of claim 36, wherein the tip is an apex having a tip is conically shaped and has a tip of radius of about 1 μm.

41. The pyrolyzed microneedle of claim 36, wherein the microneedle has Young's modulus of about 9 GPa.

42. The pyrolyzed microneedle of claim 36, wherein the microneedle has a Weibull modulus of about 3.

43. The pyrolyzed microneedle of claim 36, wherein the microneedle has a characteristic strength a of 710 MPa.
